(19) United States
(12) Patent Application Publication
(10) Pub. No.: US 2020/0315918 A1
(43) Pub. Date: Oct. 8, 2020

(54) MEDICAL VESSELS WITH VALVES AND VALVE LOCK-UNLOCK MECHANISMS

(71) Applicant: Mikael NAYGAUZ, Forrest Hills, NY (US)

(72) Inventor: Mikael NAYGAUZ, Forrest Hills, NY (US)

(21) Appl. No.: 16/439,851

(22) Filed: Jun. 13, 2019

Related U.S. Application Data
(60) Provisional application No. 62/828,473, filed on Apr. 3, 2019.
(60) Provisional application No. 62/830,453, filed on Apr. 7, 2019.
(60) Provisional application No. 62/833,791, filed on Apr. 15, 2019.
(60) Provisional application No. 62/838,944, filed on Apr. 26, 2019.
(60) Provisional application No. 62/851,454, filed on May 22, 2019.
(60) Provisional application No. 62/857,260, filed on Jun. 4, 2019.

(51) Int. Cl. A61J 1/20; A61M 39/22

(52) U.S. Cl. CPC A61J 1/2096; A61J 1/10; A61M 39/22; A61J 1/2037

(57) ABSTRACT

The present invention relates, in some embodiments thereof, to devices, systems and methods for establishing a fluid passageway between vessels. In some embodiments, the invention provides a system for the engagement and establishment of fluid passageway between vessels, the system comprises a first vessel comprising a valve; and a second vessel configured to be coupled to the first vessel; wherein the second vessel comprises a valve unlock mechanism configured to unlock/open the valve, and wherein at about the time of or following the engagement between the first and second vessels, the valve unlock mechanism unlocks/opens the valve, allowing a fluid transfer between the vessels.

### CROSS REFERENCE TO RELATED APPLICATIONS

The present application claims priority to U.S. Provisional Patent Application Ser. No. 62/828,473, entitled SYSTEM, CONTAINER, AND METHOD FOR UNLOCKING PLUNGERS OF MODULARLY ASSEMBLED MEDICAMENT CONTAINERS and filed on Apr. 3, 2019; 62/830,453, entitled SYSTEM AND METHOD FOR UNLOCKING PIERCING MEMBERS OF CONTAINERS and filed on Apr. 7, 2019; 62/833,791 entitled SYSTEMS, DEVICES AND METHODS FOR LOCKING AND UNLOCKING VALVES OF MEDICINAL VESSELS and filed on Apr. 15, 2019; 62/838,944, entitled SYSTEMS, CONTAINERS, AND METHODS HAVING PLUNGER LOCKING-UNLOCKING MECHANISMS and filed on Apr. 26, 2019; 62/851,454 entitled PLUNGER AND NEEDLE LOCKING AND UNLOCKING SYSTEMS and filed on May 22, 2019; and 62/857,260 entitled MEDICAL VESSELS WITH VALVES AND VALVES LOCK-UNLOCK MECHANISMS and filed on Jun. 4, 2019 the entire contents of which are expressly incorporated herein by reference.

All of the following United States Patent Applications are hereby incorporated herein by reference in their entireties: Ser. Nos. 16/100,840; 16/100,860; 16/100,874, 16/100,903; 16/169,695; 16/100,964; 16/100,969; 16/100,976; 16/100,981; 16/358,434; 16/358,389; 16/423,120; and 16/423,122.

### FIELD OF THE INVENTION

The present invention relates, in some embodiments thereof, to devices, systems and methods for establishing a fluid passageway between vessels. More particularly, the present invention relates, in one or more embodiments, to systems, devices, containers, and methods for the establishment of fluidic communication between vessels and for the prevention of spillage of beneficial substances prior to establishment of the fluidic communication between the vessels.

### BACKGROUND OF THE INVENTION

The medicinal practice routinely involves administration of medical substances, such as, medicaments, fluids, nutrition substances and the alike, to patients. The preparation and/or administration of such medicinal substances typically involves one or more transfers of those substances between vessels (such as, vials, syringes, infusion lines, etc.).

Additionally, beneficial substances used in medical settings such as medicaments, nutritional substances, and the alike are provided in standard commercially available amounts. This poses a disadvantage for preparing tailored and/or customized final dosage amounts for patients. The preparation of customized amounts of intracorporeally administered beneficial substances requires the manual manipulation of the beneficial substances typically by healthcare personnel. Manual manipulation involves drawing out from vials using syringes and needles of amounts of beneficial substances, measuring visually the amount of the beneficial substances that has been drawn into a syringe, and injecting the amount of the beneficial substance from a syringe into a second container, typically a bag or a bottle. The preparation of customized amount of beneficial substances typically involves using less or more than the amount provided in one commercially available container. Customized dosages particularly of chemotherapeutic agents, are typically based on a patient's weight, height, and age. Therefore, the preparation of customized final amounts of beneficial substances require the manual manipulation and measurement of beneficial substances by healthcare personnel.

Another important problem that exists when intermixing beneficial substances relates to the premature and/or accidental expulsion/spillage of a beneficial substance out of a container/device prior establishment of a fluid communication between containers/devices. For example, pharmacists when mixing an injectable medicament may draw out a dose of the medicament from a vial into a syringe. Pharmacists may accidentally push the plunger of the syringe before the syringe is engaged/connected to a second container such as a bag/bottle which would result in the expulsion/spillage of the medicament out of the syringe and into the environment and exposure of the pharmacist to the medicament. This is particularly dangerous if the medicament is a toxic substance such as a chemotherapeutic medicament which would result in the pharmacist being exposed to a toxic substance. Currently, there is no means by which to prevent the accidental expulsion of a beneficial substance out of a syringe into the environment.

Yet another problem is associated with contamination of medical substances when intermixing beneficial substances. Typically, the action of fluid transfer exposes connection interfaces/surfaces of medical vessels to the environment and accordingly the medical substances themselves to contaminants present in ambient air particles (e.g., bacteria, viruses, funguses, spores, pyrogens). In addition, connection interfaces/surfaces of medical substances are further prone to contaminations due to physical contact of the interfaces/surfaces, for example, with nonsterile gloves, or devices. Such contaminations are a major problem in the healthcare setting since those, once invading within medicinal substances, may pose substantial danger to patients.

Taken together, the above described systems and methods are either cumbersome and expensive or inefficient in addressing the problem of intermixing customized amounts of beneficial substances while preventing the premature/accidental expulsion/spillage of the beneficial substances prior to establishment of a fluidic communication between containers/devices. Thus, there is a long felt and unmet need in systems, devices and/or methods that afford the safe transfer of medical substances while preventing the spillage of medical substances and exposure of healthcare personnel to toxic substances.

### SUMMARY OF THE INVENTION

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

The present invention pertains to devices, systems and methods for establishing a fluid communication between vessels in a secure manner and while allowing a fluidic transfer when vessels attach to each other. It is an object of the present invention, in some embodiments, to provide systems, devices, and methods of preventing spillage of beneficial substances/medicaments prior to engagement of vessels. It is a further object of the present invention, in some embodiments, to provide systems, methods, containers, syringes, cartridges, and devices having valve locking and/or unlocking mechanisms that prevent the spillage of beneficial substances/medicaments/nutritional substances prior to engagement of vessels/containers.

Thus, the present invention provides a system for the engagement and establishment of fluid passageway between vessels, the system comprising:
a first vessel comprising a valve; and
a second vessel configured to be coupled to the first vessel;
wherein the first and/or second vessel(s) comprise(s) a valve unlock mechanism configured to unlock/open the valve, and wherein at about the time of or following the engagement between the first and second vessels, the valve unlock mechanism unlocks/opens the valve, allowing a fluid transfer from the first vessel into the second vessel.

In one or more embodiments, the first vessel is a container and the second vessel is a container.

In one or more embodiments, the first vessel is a syringe and the second vessel is a bag or a bottle. In one or more embodiments, the syringe has a plunger. In one or more embodiments, the plunger is a moveable wall.

In one or more embodiments, the first vessel is a cartridge and the second vessel is a container. In one or more embodiments, the container is a bag. In one or more embodiments, the container is a bottle.

In another aspect the present invention provides a system for the engagement and establishment of fluid passageway between vessels, the system comprising:
a first vessel;
a second vessel; and
a decontamination device configured to be coupled to the first and second vessels;
wherein at least one of the first vessel and second vessel includes a valve and wherein the decontamination device includes a valve unlock mechanism such that at about the time of or following the engagement between the first and/or second vessels and the decontamination device, the valve unlock mechanism unlocks/opens the valve, allowing a fluid transfer between the first and second vessels.

In another aspect the present invention provides a system for the engagement and establishment of fluid passageway between vessels, the system comprising:
a first vessel;
a second vessel; and
a decontamination device configured to be coupled to the first and second vessels;
wherein the decontamination device includes a valve and wherein at least one of the first vessel and second vessel includes a valve unlock mechanism such that at about the time of or following the engagement between the first and/or second vessels and the decontamination device, the valve unlock mechanism unlocks/opens the valve, allowing a fluid transfer between the first and second vessels.

In another aspect the present invention provides a system for the engagement and establishment of fluid passageway between vessels, the system comprising:
a first vessel;
a second vessel configured to be coupled to the first vessel; and
a valve lock mechanism disposed on the first vessel and configured to lock a valve thereof and prevent the premature fluid transfer from the first vessel to the second vessel prior to an engagement with the second vessel, wherein the first and/or second vessel(s) comprise(s) a valve unlock mechanism configured to unlock the lock mechanism of the first vessel, and wherein at the time of or following the engagement between the first and second vessels, the unlock mechanism unlocks the lock mechanism of the valve, allowing opening thereof and thereby a fluid transfer between the vessels.

In one or more embodiments, the valve is a one-way valve. In one or more embodiments, the valve is a two-way valve. In one or more embodiments, the valve is selected from a ball valve, a butterfly valve, choke valve, diaphragm valve, a gate valve, a knife valve, a needle valve, a pinch valve, a piston valve, a plug valve, a solenoid valve, a spool valve, and a combination thereof.

In one or more embodiments, valve lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the valve unlock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, a slot, and a combination thereof.

In one or more embodiments, the valve is pivotally connected to a side wall of the first and/or second vessel and moveable about said pivot. In one or more embodiments, the valve lock and/or unlock mechanism is pivotally connected to a side wall via a hinge mechanism of the first and/or second vessel and moveable about said pivot. In one or more embodiments, the valve lock and/or unlock mechanism is spring loaded or attached to a spring, allowing planar movement thereof.

In one or more embodiments, the system further comprising an engagement mechanism to connect the first vessel to the second vessel. In one or more embodiments, the engagement mechanism is selected from a thread, a luer, a ratchet teeth mechanism, a retention member, an adhesive mechanism, a clamping member and combinations thereof.

In one or more embodiments, the system further comprising a piercing member to pierce a seal/cover of the first and/or second vessel(s). In one or more embodiments, the system further comprising a piercing member lock mechanism to prevent premature seal/cover piercing of the first vessel and/or second vessel, by the piercing member.

In one or more embodiments, the system further comprising a plunger disposed on the first and/or second vessel(s), the plunger configured to allow for the fluid transfer from the first vessel into the second vessel.

In one or more embodiments, the system further comprises a plunger lock mechanism configured to lock a plunger and prevent fluid transfer from the first vessel to the second vessel prior to an engagement with the second vessel.

In one or more embodiments, the vessel is selected from the group consisting of a bottle, a vial, a bag, a syringe, an infusion line, a connector, a filter, a manifold, a bag port, a bottle port, a vial port, a cartridge and a combination thereof. In one or more embodiments, the second vessel includes a port having peripheral walls enclosing a cavity, the cavity configured to receive or engage with the first vessel, or a portion thereof. In one or more embodiments, the first vessel is a cartridge. In one or more embodiments, the first vessel is a syringe. In one or more embodiments, the first vessel is a bottle. In one or more embodiments, the bottle is a vial. In one or more embodiments, the second vessel includes a port having peripheral walls enclosing a cavity, the cavity is sized and shaped to accommodate the cartridge.

In one or more embodiments, the system further comprising a decontamination device configured to be coupled to the first vessel and second vessel and allow the fluid passageway therebetween, the decontamination device further configured to decontaminate a surface of at least one of the first and second vessels. In one or more embodiments, the decontamination device is further configured to decontaminate at least a portion of a surface of at least one of the first and second vessels.

In one or more embodiments, the decontamination device further comprises an engagement mechanism to connect the decontamination device to the first and/or second vessel. In one or more embodiments, the vessel and/or decontamination device includes a port having peripheral walls enclosing a cavity, the cavity configured to receive or engage with a vessel and/or decontamination device, or a portion thereof.

In one or more embodiments, the decontamination device is pre-attached to the first or second vessels. In one or more embodiments, the decontamination device is integrally attached to the first or second vessel. In one or more embodiments, the decontamination device forms a unitary structure with the first vessel or the second vessel. In one or more embodiments, the decontamination device includes a wiping member configured to wipe off a surface of a vessel.

In one or more embodiments, the first and/or second vessel is configured to move within the device and wherein the wiping member configured to remove contaminants from a surface of said first and/or second vessels at about the time of said vessel's movement. In one or more embodiments, at least a portion of the first and/or second vessel is configured to move within the decontamination device and wherein the wiping member is configured to remove contaminants from a surface of said first and/or second vessels at about the time of said vessel's movement.

In one or more embodiments, the wiping member is configured to move within the device such to allow decontamination a surface of at least one of said first and second vessels at about the time of said wiping member's movement. In one or more embodiments, the wiping member is configured to move within the device such to allow decontamination of a portion of a surface of at least one of said first and second vessel at about the time and/or after said wiping member's movement.

In one or more embodiments, the decontamination device includes a sliding mechanism located on and/or attached to said wiping member and/or said vessel and configured to allow for the movement of said wiping member and/or said vessel within said device. In one or more embodiments, the lock mechanism disposed on the first vessel and the unlock mechanism disposed on the decontamination device and/or first vessel.

In yet another aspect, the invention provides a syringe having a valve.

In yet another aspect, the invention provides a syringe having a valve and a valve locking mechanism.

In one or more embodiments, the valve locking mechanism is configured to keep the valve closed.

In one or more embodiments, the valve locking mechanism further configured to be unlocked by a container.

In one or more embodiments, the valve locking mechanism further configured to be unlocked by a container at about the time of engagement of the syringe to the container.

In one or more embodiments, the valve locking mechanism further configured to be unlocked by a container after engagement of the syringe to the container.

In one or more embodiments, the valve locking mechanism configured to be unlocked by a pushing motion and/or twisting motion of the syringe towards a container.

In one or more embodiments the valve locking mechanism is disposed inside a wall of the syringe.

In one or more embodiments, the valve locking mechanism is partially disposed inside a wall of the syringe.

In one or more embodiments, the valve locking mechanism is entirely disposed inside a wall of the syringe.

In one or more embodiments, the valve locking mechanism disposed inside the syringe.

In one or more embodiments, the valve locking mechanism is entirely partially disposed inside the syringe.

In one or more embodiments, the valve locking mechanism is partially disposed inside the syringe.

In one or more embodiments, the valve locking mechanism configured to lock the valve in a closed position and further configured to open the valve when in an unlocked position.

In yet another aspect, the invention provides a cartridge having a valve.

In yet another aspect, the invention provides a cartridge having a valve and a valve locking mechanism.

In one or more embodiments, the valve locking mechanism is configured to keep the valve closed.

In one or more embodiments, the valve locking mechanism further configured to be unlocked by a container.

In one or more embodiments, the valve locking mechanism further configured to be unlocked by a container at about the time of engagement of the cartridge to the container.

In one or more embodiments, the valve locking mechanism further configured to be unlocked by a container after engagement of the cartridge to the container.

In one or more embodiments, the valve locking mechanism configured to be unlocked by a pushing motion and/or twisting motion of the cartridge towards a container.

In one or more embodiments the valve locking mechanism is disposed inside a wall of the cartridge.

In one or more embodiments, the valve locking mechanism is partially disposed inside a wall of the cartridge.

In one or more embodiments, the valve locking mechanism is entirely disposed inside a wall of the cartridge.

In one or more embodiments, the valve locking mechanism disposed inside the cartridge.

In one or more embodiments, the valve locking mechanism is entirely partially disposed inside the cartridge.

In one or more embodiments, the valve locking mechanism is partially disposed inside the cartridge.

In one or more embodiments, the valve locking mechanism configured to lock the valve in a closed position and further configured to open the valve when in an unlocked position.

In yet another aspect, the invention provides a method for establishing a fluidic communication between vessels, the method comprising:
providing a first vessel having a valve and a valve lock mechanism, the valve lock mechanism configured to lock the valve in a closed position;
providing a second vessel; and
engaging/coupling the first and second vessels, wherein at about the time of engagement of the vessels, the valve locking mechanism is unlocked, allowing the valve to open and establish a fluidic communication between the first and second vessels.

In one or more embodiments, an airtight seal is formed between the vessels following the step of connecting/engaging the vessels, allowing a hermetically sealed connection between said first vessel and said second vessel.

In one or more embodiments, the first and/or second vessels include a valve unlock mechanism configured to unlock the lock mechanism at about the time of, at the time of, and/or following the engagement between the vessels.

In one or more embodiments, the step of connecting the vessels includes unlocking the lock mechanism. In one or more embodiments, the step of unlocking the lock mechanism occurs via a push motion, a twist motion, a turn motion or a combination thereof.

In one or more embodiments, the connection/engagement of the vessels occurs via a push motion, a twist motion, a turn motion or a combination thereof.

In one or more embodiments, the connection/engagement of the vessels is selected from a twisting of a thread onto a complementary thread, attaching ratchet teeth to a complementary retention member, and adhering a surface of a first vessel to the surface of said second vessel via an adhesive, and combinations thereof.

In one or more embodiments, the method further comprises actuating a piercing member/needle to pierce a vessel's surface. In one or more embodiments, the method further comprises pushing a plunger of the first vessel towards the second vessel.

In one or more embodiments, the method further comprises providing a decontamination device having a wiping member, the wiping member configured to decontaminate a surface of the first and/or second vessel(s). In one or more embodiments, the wiping member is disposed inside the decontamination device.

In one or more embodiments, the method further comprises actuating the wiping member to move within the device, thereby decontaminating the surface of the first and/or second vessel.

In one or more embodiments, the method further comprises moving the first vessel and/or second vessel within the device and via the wiping member, thereby decontaminating the surface of the first and/or second vessel. In one or more embodiments, the method further comprises moving at least a portion of at least one of the first and second vessel within the device and via the wiping member, thereby decontaminating the surface of the first and/or second vessel.

In one or more embodiments, the method further comprises transferring a fluid substance between the vessels after engaging/connecting the vessels. In one or more embodiments, the method further comprises transferring a fluid substance within the at least one vessel through the device after the surface of the at least one vessel has been decontaminated.

The invention provides in a yet another aspect a system for the engagement and establishment of fluid passageway between vessels, the system comprising:
a first vessel having a plunger and a valve;
a second vessel configured to be coupled to the first vessel; and
a plunger locking mechanism disposed on the first vessel and configured to lock the plunger thereof and prevent the plunger from expelling the content of the first vessel prior to an engagement with the second vessel, wherein the first and/or second vessel(s) comprise a plunger unlocking mechanism configured to unlock the plunger lock mechanism of the first vessel, and wherein at the time of or following the engagement between the first vessel and the second vessel, the plunger unlocking mechanism unlocks the plunger lock mechanism of the plunger allowing movement thereof and thereby a fluid transfer from the first vessel into the second vessel, the first vessel further comprising a valve locking mechanism disposed on the first vessel and configured to lock the valve thereof and prevent the valve from opening and allowing the contents of the first vessel to be expelled from the first vessel prior to engagement of the first vessel to the second vessel, wherein the first and/or second vessel(s) further comprising a valve unlocking mechanism configured to unlock the valve lock mechanism of the first vessel, and wherein at the time of or following the engagement between the first vessel and the second vessel, the valve unlocking mechanism unlocks the valve locking mechanism of the valve allowing movement thereof and thereby a fluid transfer from the first vessel into the second vessel.

In one or more embodiments, the first vessel is a container and the second vessel is a container. In one or more embodiments, the first vessel is a syringe and the second vessel is a bottle. In one or more embodiments, the first vessel is a syringe and the second vessel is a bag. In one or more embodiments, the first vessel is a cartridge and the second vessel is a bottle. In one or more embodiments, the first vessel is a cartridge and the second vessel is a bag. In on or more embodiments, the first vessel is a cartridge and the second vessel is a container. In one or more embodiments, the first vessel is a container having a plunger and the second vessel is a bottle. In one or more embodiments, the first vessel is a container having a plunger and the second vessel is a bag. In one or more embodiments, the first vessel is a container having a plunger and the second vessel is a container. In one or more embodiments, the first vessel is a vial and the second vessel is a container. In one or more embodiments, the first vessel is a container having moveable wall and the second vessel is a container. In one or more embodiments, the moveable wall is a plunger. In one or more embodiments, the first container is a syringe and the second container is a connector. In one or more embodiments, the first container is a syringe and the second container is a manifold. In one or more embodiments, the first container is a cartridge and the second container is a connector. In one or more embodiments, the first container is a cartridge and the second container is a manifold.

In one or more embodiments, the plunger lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the plunger unlock mechanism is selected from the group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, a slot, and a combination thereof.

In one or more embodiments, the plunger lock mechanism is moveable between a locked position wherein the lock mechanism protrudes from a side wall of the first vessel such to disrupt movement forward of the plunger and an unlocked retracted position wherein the plunger is capable of moving forward thereby expelling the substance in the first vessel.

In one or more embodiments, the lock mechanism is pivotally connected to a side wall of the first vessel and moveable about the pivot.

In one or more embodiments, the lock mechanism is spring loaded or attached to a spring, allowing planar movement thereof.

In one or more embodiments, the lock mechanism includes a retractable ball which can protrude into an inner lumen of the vessel thereby lock the plunger or retract to a socket in a side wall of the first and/or second vessel, thereby unlock the plunger.

In one or more embodiments, the lock mechanism includes a magnetic ball which can protrude into an inner lumen of the vessel thereby lock the plunger or retract to a magnetic socket in a side wall of the first and/or second vessel, thereby unlock the plunger.

In one or more embodiments, the unlock mechanism includes an elongated arm disposed in the second vessel and configured to unlock the lock mechanisms.

In one or more embodiments, the first and/or second vessel include(s) a port having peripheral walls enclosing a cavity, the cavity configured to receive or engage with a vessel, or a portion thereof. In one or more embodiments, the lock mechanism and/or the unlocking mechanism disposed on an internal and/or external position of the port. In one or more embodiments, the first and/or second vessel includes a port having peripheral walls enclosing a cavity, the cavity configured to receive or engage with a vessel, or a portion thereof, wherein the engagement mechanism disposed on an internal and/or external position of the port. In one or more embodiments, the first or second vessels configured to be coupled to a plurality of vessels.

In one or more embodiments, the lock mechanism disposed on the first vessel and the unlock mechanism disposed on the decontamination device and/or first vessel.

According to yet another aspect the present invention provides a method of establishing a fluid passageway between vessels, the method comprises the steps of:
providing a first vessel and a second vessel; wherein the first vessel comprises a plunger and a plunger lock mechanism and the first and/or second vessel(s) includes a plunger unlock mechanism; wherein the plunger lock mechanism configured to lock the plunger thus preventing the plunger from expelling a content within the first vessel; and wherein the plunger unlock mechanism configured to unlock the plunger lock mechanism;
connecting the first vessel to the second vessel;
unlocking the plunger lock mechanism; and
pushing a plunger of the first vessel towards the second vessel to thereby allow the fluid passageway between the vessels.

In one or more embodiments, the method further comprising a valve and a valve locking mechanism disposed on the first vessel, the valve locking mechanism configured to be unlocked by the second vessel. In one or more embodiments, the method further comprising unlocking of the valve locking mechanism at about the time of engagement of the first vessel to the second vessel. In one or more embodiments, the method further comprising unlocking of the valve locking mechanism at the time of engagement of the first vessel to the second vessel. In one or more embodiments, the method further comprising unlocking of the valve locking mechanism after the engagement of the first and second vessels.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B are side cut view illustrations demonstrating a syringe with a valve disposed within a tip of the syringe, according to some embodiments of the invention.

FIGS. 2A-2B are side cut view illustrations demonstrating a syringe with a valve disposed about the surface of a tip of the syringe, according to some embodiments of the invention.

FIGS. 3A-3B are side cut view illustrations demonstrating a cartridge with a valve disposed within an aperture of the cartridge, according to some embodiments of the invention.

FIGS. 4A-4B are side cut view illustrations demonstrating a cartridge with a valve disposed about an inner surface of an aperture of the cartridge, according to some embodiments of the invention.

FIGS. 5A-5B are side cut view illustrations demonstrating a cartridge with a valve disposed about an outer surface of an aperture of the cartridge, according to some embodiments of the invention.

FIGS. 6A-6B are side cut view illustrations demonstrating an infusion bag with a valve disposed about an inner surface of an aperture of the bag, according to some embodiments of the invention.

FIGS. 7A-7B are side cut view illustrations demonstrating an infusion bag with a valve disposed about an exterior position of an aperture of the bag, according to some embodiments of the invention.

FIGS. 8A-8B are side cut view illustrations demonstrating a cartridge having a valve and a valve lock mechanism in the form of poles, according to some embodiments of the invention.

FIGS. 9A-9B are side cut view illustrations demonstrating a cartridge having a valve and a valve lock mechanism in the form of pins, according to some embodiments of the invention.

FIGS. 10A-10B are side cut view illustrations demonstrating a cartridge having a valve and a valve lock mechanism in the form of hooks, according to some embodiments of the invention.

FIGS. 11A-11B are side cut view illustrations demonstrating a cartridge having a valve and a valve lock mechanism in the form of latches, according to some embodiments of the invention.

FIGS. 12A-12B are side cut view illustrations demonstrating a system having a first vessel, and a second vessel, the first vessel in the form of a cartridge having a valve, a valve lock mechanism in the form of hooks, and a valve unlock mechanism having elongated arms, according to some embodiments of the invention.

FIGS. 13A-13B are side cut view illustrations demonstrating a system having a first vessel, and a second vessel, the first vessel in the form of a cartridge having a valve, a valve lock mechanism in the form of latches, and a valve unlock mechanism having elongated arms, according to some embodiments of the invention.

FIGS. 14A-14B are side cut view illustrations demonstrating a system having a first vessel and a second vessel, the first vessel in the form of a syringe having a valve extending through the side walls of a tip of the syringe and protruding externally therefrom, according to some embodiments of the invention.

FIGS. 15A-15B are side cut view illustrations demonstrating a system having a first vessel and a second vessel, the first vessel in the form of a cartridge having a valve, the second vessel includes a valve unlock mechanism with tenons configured to enter sockets of the valve, according to some embodiments of the invention.

FIG. 16 is a side cut view illustration demonstrating a system with a plurality of first vessels that can be engaged with a second vessel, the first vessels include valves and the second vessel includes a valve unlock mechanism in the form of tenons, according to some embodiments of the invention.

FIG. 17 is a side cut view illustration demonstrating a system with a plurality of first vessels that can be engaged with a second vessel, the second vessel includes valves and the first vessels include a valve unlock mechanism in the form of tenons, according to some embodiments of the invention.

FIGS. 18A-18D are side cut view illustrations demonstrating a system with a first vessel in the form of a cartridge, a second vessel in the form of an infusion bag, and a decontamination device, the first vessel having a valve and the decontamination device has a valve unlock mechanism in the form of a moveable ball, according to some embodiments of the invention.

FIG. 19 is a side cut view illustration demonstrating a system with optional first vessels, optional second vessels and a decontamination device in the form of a connector, the vessels include a valve and a valve lock mechanism and the decontamination device includes a valve unlock mechanism, according to some embodiments of the invention.

FIG. 20 is a side cut view illustration demonstrating a system with optional first vessels, optional second vessels and a decontamination device in the form of a connector, the connector includes valves and valve lock mechanisms on ports thereof, the first and second vessels include valve unlock mechanisms, according to some embodiments of the invention.

FIGS. 21A-21B are side cut view illustrations demonstrating a system with a first vessel, a second vessel and a decontamination device, the decontamination device includes a wiping member configured to move within the device and decontaminate the surfaces of the vessels, the first and second vessels each includes a valve for fluid flow monitoring, according to some embodiments of the invention.

FIG. 22 is a side cut view illustration demonstrating a system with a first vessel, a second vessel and a decontamination device, the decontamination device includes a moveable wiping member, the first and second vessels each includes a valve and a valve lock mechanism, according to some embodiments of the invention.

FIGS. 23A-23B are a side cut view illustrations demonstrating a system with a first vessel, a second vessel and a decontamination device having a moveable wiping member, the first vessel includes a valve and a valve lock mechanism and the decontamination device includes a valve unlock mechanics in the form of a retractable ball, according to some embodiments of the invention.

FIGS. 24A-24B are a side cut view illustrations demonstrating a system with a first vessel, a second vessel and a decontamination device having a moveable wiping member, the decontamination device further includes a valve on a port thereof, and the first vessel includes a valve unlock mechanism, according to some embodiments of the invention.

FIGS. 25A-25B are a side cut view illustrations demonstrating a system with a first vessel, a second vessel and a decontamination device having a moveable wiping member, the decontamination device further includes a valve on a port thereof, and the first vessel includes a valve unlock mechanism, according to some embodiments of the invention.

FIGS. 26A-26B are a side cut view illustrations demonstrating a system with a first vessel, a second vessel and a decontamination device having a moveable wiping member, the decontamination device further includes downward extending walls enclosing a lumen for accommodating the first vessel, the first vessel include a valve and the port of the device includes a valve unlock mechanism, according to some embodiments of the invention.

FIGS. 27A-27B are a side cut view illustrations demonstrating a system with a first vessel, a second vessel and a decontamination device having a moveable wiping member, the decontamination device further includes downward extending walls enclosing a lumen for accommodating the first vessel, the first vessel includes a valve, and a valve lock mechanism, a valve unlock mechanism is disposed on the downward extending walls of the decontamination device, according to some embodiments of the invention.

FIGS. 28A-28C are a side cut view illustrations demonstrating a system with a first vessel, a second vessel and a decontamination device wherein at least the first vessel is moveable within the device, the decontamination device further includes downward extending walls enclosing a lumen for accommodating the first vessel, the first vessel includes a valve, and a valve lock mechanism, a valve unlock mechanism in the form of a retractable ball is disposed on the downward extending walls of the decontamination device, according to some embodiments of the invention.

FIGS. 29A-29B are a side cut view illustrations demonstrating a system with a first vessel, a second vessel and a decontamination device having one or more discs, the decontamination device further includes downward extending walls enclosing a lumen for accommodating the first vessel, the first vessel includes a valve, and a valve lock mechanism, a valve unlock mechanism in the form of a retractable ball is disposed on the downward extending walls of the decontamination device, according to some embodiments of the invention.

FIG. 30 is a side cut view illustration demonstrating a system with optional first vessels, optional second vessels and a decontamination device in the form of a connector that allows movement of the first and/or second vessels, the first and/or second vessel(s) include(s) a valve and a valve lock mechanism and the decontamination device includes a valve unlock mechanism, according to some embodiments of the invention.

FIG. 31 is a side cut view illustration demonstrating a system with optional first vessels, optional second vessels and a decontamination device in the form of a connector that allows movement of the first and/or second vessels, the connector includes vertical extending walls for accommodating a vessel, the first and/or second vessel(s) include(s) a valve and a valve lock mechanism and the decontamination device includes a valve unlock mechanism, according to some embodiments of the invention.

FIG. 32 is a side cut view illustration demonstrating a system with optional first vessels, optional second vessels and a decontamination device in the form of a connector that allows movement of the first and/or second vessels, the connector includes valves, valve lock mechanisms and vertical extending walls for accommodating a vessel, the first and/or second vessel(s) include(s) a valve unlock mechanism, according to some embodiments of the invention.

FIGS. 33A-33B are side cut view illustrations demonstrating a syringe that includes a valve and a valve lock mechanism, a plunger with a plunger lock and unlock mechanisms and a piercing member with a piercing member lock mechanism.

FIGS. 34A-34B are side cut view illustration demonstrating a system with a first vessel in the form of a syringe, and a second vessel in the form of a container, the syringe includes a valve and a valve lock mechanism, a plunger with a plunger lock and unlock mechanisms and a piercing member with a piercing member lock mechanism.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary valve lock and unlock mechanisms for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention pertains to devices, methods and systems for fluid transfer between one or more vessels in a secure manner. The present invention pertains to systems, devices, and methods for preventing the premature spillage of beneficial substances/medicaments from containers intended for use in a modular assembly format. The invention provides a solution to an unmet and long felt need in the medical settings and provides vessels with one or more valves and valve lock, and optionally unlock mechanisms, thereby preventing spillage of medical substances. In one or more embodiments, the herein disclosed devices and systems pertain to valve locking and unlocking mechanisms for fluid transfer between vessels. In one or more embodiments, the herein disclosed devices and systems pertain to valve locking and unlocking mechanisms for securing and monitoring a fluid transfer between vessels.

Thus, it is an object of the present invention, in some embodiments, to provide systems, devices, and methods for establishing fluidic communications between containers. It is an object of the present invention, in some embodiments, to provide vessels having one or more valves for fluid transfer monitoring. It is an object of the present invention, in some embodiments, to provide vessels having at least one valve locking mechanisms. It is an object of the present invention, in some embodiments, to provide vessels having at least one valve unlocking mechanisms. It is an object of the present invention, in some embodiments, to provide systems, devices, and methods of preventing spillage of beneficial substances/medicaments prior to the engagement of at least two vessels.

The present invention pertains to a system with at least two containers/vessels for establishing a fluidic communication therebetween at about the time, at the time, and/or following an engagement/coupling of the at least two containers/vessels. In some embodiments, at least one of the containers/vessels is having a valve configured to be opened by a second container/vessel, wherein upon opening of the valve a fluidic communication can be established between the containers/vessels.

In one or more embodiments, engaging/coupling the vessels mediates the unlocking/opening of valves, allowing for a fluidic communication between the vessels. Advantageously, the invention provides systems capable of preventing premature leakage of contents of the containers into the environment. Hence, the system prevents exposure of healthcare personnel to optionally toxic substances which may be contained within the vessels, providing safe means of establishing fluidic communication between at least two containers.

The valve is configured to be operable in at least two positions, a first closed position which prevents the flow of a substance into and/or out of the vessel, and a second open position which allows the flow of a substance into and/or out of the vessel.

Thus, in one or more embodiments, the invention provides a system for establishing fluidic communication between vessels, the system comprising:
a first vessel having a valve; and
a second vessel;
wherein upon the engagement between the first and second vessels, the valve is opened allowing a fluid communication between the vessels.

In one or more embodiments, the invention provides a system for establishing fluidic communication between vessels, the system comprising:
a first vessel having a valve and a valve lock mechanism; and
a second vessel;
wherein upon the engagement between the first and second vessels, the valve lock mechanism is opened allowing opening of the valve and a fluid communication between the vessels.

In one or more embodiments, the invention provides a system for establishing fluidic communication between vessels, the system comprising:
a first vessel having a valve and a valve lock mechanism; and
a second vessel;
wherein the first and/or second vessel includes a valve unlock mechanism, wherein upon the engagement between the first and second vessels, the valve unlock mechanism unlocks the valve lock mechanism, allowing opening of the valve and a fluid communication between the vessels.

In one or more embodiments, the invention provides a system for establishing fluidic communication between vessels, the system comprising:
a first vessel having a valve;
a second vessel; and
a decontamination device configured to be coupled to the first and second vessels, the decontamination device allowing a fluid communication between the vessels and decontaminating a surface of the first and/or second vessels;
wherein upon the engagement between the first vessel and the decontamination device, the valve is opened allowing the fluid communication between the vessels.

In one or more embodiments, the invention provides a system for establishing fluidic communication between vessels, the system comprising:
a first vessel having a valve and a valve lock mechanism;
a second vessel; and
a decontamination device configured to be coupled to the first and second vessels, the decontamination device allowing a fluid communication between the vessels and decontaminating a surface of the first and/or second vessels;
wherein upon the engagement between the first vessel and the decontamination device, the valve lock mechanism is unlocked allowing opening of the valve and a fluid communication between the vessels.

In one or more embodiments, the invention provides a system for establishing fluidic communication between vessels, the system comprising:
a first vessel having a valve and a valve lock mechanism;
a second vessel; and
a decontamination device configured to be coupled to the first and second vessels, allow a fluid communication between the vessels and decontaminate a surface of the first and/or second vessels;
wherein the first vessel and/or decontamination device include(s) a valve unlock mechanism, wherein upon the engagement between the first vessel and the decontamination device, the valve unlock mechanism unlocks the valve locking mechanism, allowing opening of the valve and a fluid communication between the vessels.

In one or more embodiments, the second vessel further comprises a valve. In one or more embodiments, the valve of the second vessel is connected/engaged/coupled to a valve locking mechanism. In one or more embodiments, the second vessel includes a valve and a valve lock mechanism, and the first vessel includes a valve unlocking mechanism configured to unlock the valve locking mechanism of the second vessel.

Various forms of the valve locking mechanism are contemplated and applicable, including without limitation a hook, pin, retractable pin, locking key, latch, knob, lock, dent, hole, channel, bangle, slot, or a combination thereof. Various forms of the valve unlocking mechanism are contemplated and applicable, including without limitation a hook, pin, retractable pin, locking key, latch, knob, lock, dent, hole, channel, bangle, slot.

Various positions for the valve and/or valve lock mechanism and/or valve unlock mechanism are contemplated, such as within the vessel, within a tip or port of the vessel, on a surface of an aperture/tip/port of the vessel, on internal surfaces of side walls of the vessel. on a cavity of the vessel, extending through a wall of the vessel, and combinations thereof.

In one or more embodiments, the valve locking mechanism engages/couples with the valve unlocking mechanism at about the time of engagement/coupling of the first vessel and the second vessel.

Various motions can be applicable for opening the valve lock mechanism and/or the valve. In one or more embodiments, the unlocking of the valve locking mechanism is mediated via a pushing motion of the second vessel towards the first vessel. In one or more embodiments, the unlocking of the valve locking mechanism is mediated via a pushing motion of the first vessel towards the second vessel. In one or more embodiments, the unlocking of the valve locking mechanism is mediated via a pushing motion of the first and second vessels towards each other.

In one or more embodiments, the unlocking of the valve locking mechanism is mediated via a twisting motion of the first vessel in relation to the second vessel. In one or more embodiments, the unlocking of the valve locking mechanism is mediated via a twisting motion of the second vessel in relation to the first vessel. In one or more embodiments, the unlocking of the valve locking mechanism is mediated via a twisting motion of the first and second vessels in relation to each other.

In one or more embodiments, the unlocking of the valve locking mechanism is mediated via a turning motion of the first vessel in relation to the second vessel. In one or more embodiments, the unlocking of the valve locking mechanism is mediated via a turning motion of the second vessel in relation to the first vessel. In one or more embodiments, the unlocking of the valve locking mechanism is mediated via a turning motion of the first and second vessels in relation to each other.

In one or more embodiments, the unlocking of the valve locking mechanism is mediated via a push and twist motion of the second vessel in relation to the first vessel. In one or more embodiments, the unlocking of the valve locking mechanism is mediated via a push and twist motion of the first vessel in relation the second vessel. In one or more embodiments, the unlocking of the valve locking mechanism is mediated via a push and twist motion of the first and second vessels in relation to each other.

In one or more embodiments, the unlocking of the valve locking mechanism is mediated via a twist and push motion of the second vessel in relation to the first vessel. In one or more embodiments, the unlocking of the valve locking mechanism is mediated via a twist and push motion of the first vessel in relation the second vessel. In one or more embodiments, the unlocking of the valve locking mechanism is mediated via a twist and push motion of the first and second vessels in relation to each other.

Optionally, the valve locking mechanism is configured to be unlocked by a pushing motion of the first vessel towards the second vessel. Alternatively, the valve locking mechanism is configured to be unlocked by a pushing motion of the second vessel relative to the first vessel. Alternatively, the valve locking mechanism is configured to be unlocked by a pushing motion of the first and second vessels towards each other.

In one or more embodiments, at least one of a push and twist motions moves the valve locking mechanism from a locked position where the valve is unable to move to an unlocked position where the valve can move forward. In one or more embodiments, at least one of a push and twist motions moves the valve locking mechanism from a locked position where the valve is closed to an unlocked position where the valve is open and allows fluid to pass through the valve.

The present invention further provides a method for establishing a fluidic communication between vessels, the method comprising:
providing a first vessel having a valve;
providing a second vessel; and
engaging/coupling the first and second vessels, wherein at about the time of engagement of the vessels, the valve opens, allowing the valve to open and establishment of a fluidic communication between the first and second vessels.

The present invention further provides a method for establishing a fluidic communication between vessels, the method comprising:
providing a first vessel having a valve and a valve lock mechanism, the valve lock mechanism configured to lock the valve in a closed position;
providing a second vessel; and
engaging/coupling the first and second vessels, wherein at about the time of engagement of the vessels, the valve lock mechanism is unlocked, allowing the valve to open and establishment of a fluidic communication between the first and second vessels.

The present invention further provides a method for establishing a fluidic communication between vessels, the method comprising:
providing a first vessel having a valve and a valve lock mechanism, the valve lock mechanism configured to lock the valve in a closed position;
providing a second vessel having a valve unlock mechanism, the valve unlocking mechanism configured to unlock the valve of the first vessel;
engaging/coupling the first and second vessels, wherein at about the time of engagement of the vessels, the valve locking mechanism is unlocked, allowing the valve to open and establishment of a fluidic communication between the first and second vessels.

In one or more embodiments, the herein disclosed devices and systems include a vessel with a plunger and a plunger lock mechanism that can lock the plunger, preventing premature transfer of the content from the vessel. Various forms of the plunger lock mechanism are herein contemplated. Non-limiting examples of the plunger locking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall. The plunger lock mechanism may be moveable between a locked position and an unlocked position. Optionally, the plunger lock position includes extension of the lock mechanism into a hole/dent/socket/channel. Optionally, in the plunger lock position, the lock mechanism is extended into the hole/dent/socket/channel and in the unlock position, the lock mechanism is retracted. In one or more embodiments, the vessel is a syringe. In one or more embodiments, the vessel is a cartridge. In one or more embodiments, the vessel is a container.

In one or more embodiments, the herein disclosed devices and systems include a vessel having a plunger, a plunger lock mechanism, a valve, and a valve lock mechanism. In one or more embodiments, the herein disclosed devices and systems further include the vessel having a piercing member and a piercing member lock mechanism. In one or more embodiments, the piercing member is a needle. In one or more embodiments, the vessel is a selected from a syringe, a cartridge, a vial, a container, a bottle, and combinations thereof.

In one or more embodiments, the herein disclosed devices and systems include a plunger unlock mechanism that can unlock the lock mechanism of the plunger. In one or more embodiments, the plunger unlock mechanism unlocks the lock mechanism at about the time of or following an engagement between a first vessel and a second vessel. Various forms of the unlock mechanism are herein contemplated. Non-limiting examples of the unlocking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, and a slot.

In one or more embodiments, the present invention provides a vessel having a moveable wall locking mechanism. In one or more embodiments, the present invention provides a vessel having a moveable wall and a moveable wall locking mechanism. In one or more embodiments, the present invention provides a vessel having a moveable wall unlocking mechanism. In one or more embodiments, the moveable wall is configured to expel the contents of the container out of the container. In one or more embodiments, the moveable wall is a plunger. In one or more embodiments, the vessel is a bottle, a vial, a syringe, a container, and combinations thereof.

In a further aspect, the present invention provides a method for establishing fluid communication between vessels, the method comprising:
providing a first vessel having a plunger and a plunger locking mechanism;
providing a second vessel having a plunger unlocking mechanism;
connecting the first vessel to the second vessel; and
pushing a plunger of the first vessels towards the second vessel to thereby establishing a fluid passageway between the vessels, wherein the plunger locking mechanism configured to lock the plunger thus preventing the plunger from moving and expelling a substance within the first vessel prior to the engagement between the first vessel and the second vessel, wherein the plunger unlocking mechanism configured to unlock the plunger locking mechanism of the first vessel; wherein upon the connection between the plunger unlocking mechanism and the plunger locking mechanism, the plunger locking mechanism is unlocked by the plunger unlocking mechanism thus allowing the plunger to move and expel the contents of the first vessel into the second vessel.

In one or more embodiments, the unlocking of the plunger locking mechanism is mediated via a pushing motion of the second vessel towards the first vessel. In one or more embodiments, the unlocking of the plunger locking mechanism is mediated via a pushing motion of the first vessel towards the second vessel.

In one or more embodiments, the unlocking of the plunger locking mechanism is mediated via a pushing motion of the first and second vessels towards each other.

In one or more embodiments, the unlocking of the plunger locking mechanism is mediated via a twisting motion of the first vessel in relation to the second vessel. In one or more embodiments, the unlocking of the plunger locking mechanism is mediated via a twisting motion of the second vessel in relation to the first vessel. In one or more embodiments, the unlocking of the plunger locking mechanism is mediated via a twisting motion of the first and second vessels in relation to each other.

In one or more embodiments, the unlocking of the plunger locking mechanism is mediated via a turning motion of the first vessel in relation to the second vessel. In one or more embodiments, the unlocking of the plunger locking mechanism is mediated via a turning motion of the second vessel in relation to the first vessel. In one or more embodiments, the unlocking of the plunger locking mechanism is mediated via a turning motion of the first and second vessels in relation to each other.

In one or more embodiments, the unlocking of the plunger locking mechanism is mediated via a push and twist motion of the second vessel in relation to the first vessel. In one or more embodiments, the unlocking of the plunger locking mechanism is mediated via a push and twist motion of the first vessel in relation the second vessel. In one or more embodiments, the unlocking of the plunger locking mechanism is mediated via a push and twist motion of the first and second vessels in relation to each other.

In one or more embodiments, the unlocking of the plunger locking mechanism is mediated via a twist and push motion of the second vessel in relation to the first vessel. In one or more embodiments, the unlocking of the plunger locking mechanism is mediated via a twist and push motion of the first vessel in relation the second vessel. In one or more embodiments, the unlocking of the plunger locking mechanism is mediated via a twist and push motion of the first and second vessels in relation to each other.

In one or more embodiments, at least one of a push and twist motions moves the plunger locking mechanism from a locked position where the plunger is unable to move to an unlocked position where the plunger is can move forward.

In one or more embodiments, the herein disclosed devices and systems include a vessel with a piercing member configured to pierce a seal/cover/membrane of a vessel. In one or more embodiments, the herein disclosed devices and systems include a vessel with a piercing member and a piercing member lock mechanism that can lock the piercing member, preventing premature seal/cover piercing. Various forms of the piercing member lock mechanism are herein contemplated. Non-limiting examples of the piercing member locking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall. The piercing member lock mechanism may be moveable between a locked position and an unlocked position. Optionally, the piercing member lock position includes extension of the lock mechanism into a hole/dent/socket/channel. Optionally, in the piercing member lock position, the lock mechanism is extended into the hole/dent/socket/channel and in the unlock position, the lock mechanism is retracted.

In one or more embodiments, the herein disclosed devices and systems include a piercing member unlock mechanism that can unlock the lock mechanism of the piercing member. In one or more embodiments, the piercing member unlock mechanism unlocks the lock mechanism at about the time of or following an engagement between a first vessel and a second vessel. Various forms of the unlock mechanism are herein contemplated. Non-limiting examples of the unlocking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, and a slot.

In one or more embodiments, the unlocking of the piercing member locking mechanism is mediated via a pushing motion of the second vessel towards the first vessel. In one or more embodiments, the unlocking of the piercing member locking mechanism is mediated via a pushing motion of the first vessel towards the second vessel. In one or more embodiments, the unlocking of the piercing member locking mechanism is mediated via a pushing motion of the first and second vessels towards each other.

In one or more embodiments, the unlocking of the piercing member locking mechanism is mediated via a twisting motion of the first vessel in relation to the second vessel. In one or more embodiments, the unlocking of the piercing member locking mechanism is mediated via a twisting motion of the second vessel in relation to the first vessel. In one or more embodiments, the unlocking of the piercing member locking mechanism is mediated via a twisting motion of the first and second vessels in relation to each other.

In one or more embodiments, the unlocking of the piercing member locking mechanism is mediated via a turning motion of the first vessel in relation to the second vessel. In one or more embodiments, the unlocking of the piercing member locking mechanism is mediated via a turning motion of the second vessel in relation to the first vessel. In one or more embodiments, the unlocking of the piercing member locking mechanism is mediated via a turning motion of the first and second vessels in relation to each other.

In one or more embodiments, the unlocking of the piercing member locking mechanism is mediated via a push and twist motion of the second vessel in relation to the first vessel. In one or more embodiments, the unlocking of the piercing member locking mechanism is mediated via a push and twist motion of the first vessel in relation the second vessel. In one or more embodiments, the unlocking of the piercing member locking mechanism is mediated via a push and twist motion of the first and second vessels in relation to each other.

In one or more embodiments, the unlocking of the piercing member locking mechanism is mediated via a twist and push motion of the second vessel in relation to the first vessel. In one or more embodiments, the unlocking of the piercing member locking mechanism is mediated via a twist and push motion of the first vessel in relation the second vessel. In one or more embodiments, the unlocking of the piercing member locking mechanism is mediated via a twist and push motion of the first and second vessels in relation to each other.

In one or more embodiments, at least one of a push and twist motions moves the piercing member locking mechanism from a locked position where the piercing member is unable to move to an unlocked position where the piercing member can move forward.

In a further aspect, the present invention provides a container having a plunger, a plunger lock mechanism, a valve, and a valve lock mechanism. In one or more embodiments, the container further comprising a piercing member and a piercing member lock mechanism. In one or more embodiments, the piercing member is a needle. In one or more embodiments, the container is selected from a vial, a bottle, a syringe, a cartridge, and combinations thereof.

In one embodiment, the present invention further provides devices, systems and methods configured to decontaminate one or more surfaces of a vessel(s). In one or more embodiments, the devices and systems of the invention allow a substantially decontaminated fluid transfer between the one or more vessel(s). The engagement and/or fluid transfer between the one or more vessels may accordingly be utilized via the decontamination devices which decontaminate the vessel(s) and also allow the fluid transfer. A locking mechanism and/or an unlocking mechanism may be disposed on the decontamination device, and one or both engaging vessels, thereby locking and unlocking a plunger.

In a further aspect, the present invention provides a system for decontaminating a surface of a vessel, the system comprising:
a first vessel having a plunger lock mechanism, the plunger lock mechanism configured to lock a plunger within the vessel thus preventing premature movement thereof;
a second vessel; and
a decontamination device having a first and second ports for connection to the first and second vessels, the decontamination device comprising a wiper/wiping member disposed therein the device and configured to remove contaminants from a surface of at least one of the first and second vessels, the decontamination device further comprises a plunger unlock mechanism configured to unlock a plunger lock mechanism of the first vessel,
wherein upon the engagement between the first vessel and the decontamination device, the lock mechanism is unlocked, allowing a fluid transfer between the first and second vessels via the decontamination device.

In one or more embodiments, the first vessel is a syringe. In one or more embodiments, the syringe has a plunger. In one or more embodiments, the first vessel is a cartridge. In one or more embodiments, the first vessel has a moveable wall. In one or more embodiments, the moveable wall is a plunger. In one or more embodiments, decontamination device has an actuator for effecting a wiping action of the wiping member. In one or more embodiments, the actuator is a handle. In one or more embodiments, the actuator is a tab.

In a further aspect, the present invention provides a method for establishing a fluid communication between vessels, the method comprises
providing a first vessel having a plunger lock mechanism, the plunger lock mechanism configured to lock a plunger within the vessel thus preventing premature movement thereof;
providing a second vessel; and
providing a decontamination device having a first and second ports for connection to the first and second vessels, the decontamination device comprising a wiper/wiping member disposed therein the device and configured to remove contaminants from a surface of at least one of the first and second vessels, the decontamination device further comprises a plunger unlock mechanism configured to unlock a plunger lock mechanism of the first vessel,
connecting the first vessel to the decontamination device;
wherein upon the engagement between the first vessel and the decontamination device, the plunger unlock mechanism of the decontamination device unlocks the plunger lock mechanism of the first vessel, thus allowing for the movement of the plunger of the first vessel to expel the contents of the vessel into the decontamination device and thereby establish a fluid communication between the first vessel and the second vessel.

In one or more embodiments, the unlocking of the plunger lock mechanism of the first vessel occurs due to a pushing motion of the decontamination device towards the first vessel. In one or more embodiments, the unlocking of the plunger lock mechanism of the vessel occurs due to a pushing motion of the vessel towards the decontamination device. In one or more embodiments, the unlocking of the plunger lock mechanism of the vessel occurs due to a pushing motion of the vessel and the decontamination device towards each other.

In one or more embodiments, the unlocking of the plunger lock mechanism of the vessel occurs due to a twisting/turning motion of the decontamination device relative to the vessel. In one or more embodiments, the unlocking of the plunger lock mechanism of the vessel occurs due to a twisting/turning motion of the vessel relative to the decontamination device. In one or more embodiments, the unlocking of the plunger lock mechanism of the vessel occurs due to a twisting/turning motion of the vessel and the decontamination device relative to each other.

In one or more embodiments, the unlocking of the plunger lock mechanism of the vessel occurs due to a push and twist motion of the decontamination device relative to the vessel. In one or more embodiments, the unlocking of the plunger lock mechanism of the vessel occurs due to a push and twist motion of the vessel relative to the decontamination device. In one or more embodiments, the unlocking of the plunger lock mechanism of the vessel occurs due to a push and twist motion of the vessel and the decontamination device towards each other.

In one or more embodiments, the unlocking of the plunger lock mechanism of the vessel occurs due to a twist and push motion of the decontamination device relative to the vessel. In one or more embodiments, the unlocking of the plunger lock mechanism of the vessel occurs due to a twist and push motion of the vessel relative to the decontamination device. In one or more embodiments, the unlocking of the plunger lock mechanism of the vessel occurs due to a twist and push motion of the vessel and the decontamination device towards each other.

Optionally, the decontamination device includes an actuator, disposed through a wall of the device, for manipulating the movement of the wiping member, and thereby decontaminating the surface of the first and/or second vessels.

Optionally, the locking mechanism may be disposed on the vessel and/or the unlocking mechanism on the decontamination. Various positions of the locking/unlocking mechanism on the vessel and/or decontamination device are herein contemplated. The locking/unlocking mechanism may be disposed, without limitation, on a vessel's/device's body, port, aperture, wall, plunger and/or compartment. The locking mechanism may be disposed in an external and/or internal position of the vessel/device. Optionally, the decontamination device includes one or more compartments. Optionally, the decontamination device includes in a first compartment thereof a connecting structure/port for receiving a vessel. The connecting structure/port may mediate a connection with a vessel, via an engagement mechanism. Various forms of connecting structures/ports are herein contemplated. In one or more embodiments, the connecting structure/port extends downwardly from a top inner wall of a first compartment. In one or more embodiments, the connecting structure/port includes extension bodies (e.g., an elongated arm), clamping members, a threaded structure, minimized built-in dent structures, elongated extensions, and a circular body.

Various form of first and/or second vessels are contemplated. For example, the vessel may be a cartridge. In one or more embodiments, the cartridge includes protective side walls. In one or more embodiments, the moveable wall is a plunger. In one or more embodiments, the moveable wall is made of a rubber material. In one or more embodiments, the moveable wall is made of a plastic material. In one or more embodiments, the moveable wall is made of an elastomeric material. In one or more embodiments, at least one of the first and second vessels is a bag. In one or more embodiments, at least one of the first and second vessels is a bottle. In one or more embodiments, the first vessel is a bag and the second vessel is a syringe. In one or more embodiments, the first vessel is a syringe and the second vessel is a bag. In one or more embodiments, the first vessel is a bottle and the second vessel is a syringe. In one or more embodiments, the second vessel is a cartridge having a plunger.

Various types, combinations and numbers of plunger and/or valve and/or piercing member locking mechanisms may be provided in each of the herein disclosed systems and/or devices and/or container, to ensure the locking action. For example, a combination of two locking mechanisms or more may be provided in each device/system/vessel/container.

Various types, combinations and numbers of plunger and/or valve and/or piercing member unlocking mechanisms may be provided in each of the herein disclosed systems, to ensure the unlocking action. For example, a combination of two unlocking mechanisms or more may be provided in each device/system/vessel. In one or more embodiments, the locking mechanism is unlocked by the unlocking mechanism at the time of engagement of the first and second vessels. In one or more embodiments, the locking mechanism is unlocked by the unlocking mechanism after the engagement of the first and second vessels. In one or more embodiments, the locking and/or unlocking mechanism is disposed through a wall of the vessel. In one or more embodiments, the locking and/or unlocking mechanism is disposed on an interior wall of the vessel. In one or more embodiments, the locking and/or unlocking mechanism is disposed on an exterior wall of the vessel. In one or more embodiments, the locking and/or unlocking mechanism is disposed inside a wall of the vessel.

In one or more embodiments, the valve and/or plunger and/or piercing member unlock mechanism is operable to unlock a lock mechanism by displacing/removing a lock mechanism (e.g., a pin) from a channel/slot. In one or more embodiments, the valve and/or plunger and/or piercing member unlock mechanism is operable to unlock a lock mechanism by displacing/removing a lock mechanism (e.g., a stopper) from a dent. In one or more embodiments, the valve and/or plunger and/or piercing member unlock mechanism is operable to unlock a lock mechanism by displacing/removing a stopper from a protrusion of a valve/piercing member/plunger. In one or more embodiments, the valve and/or plunger and/or piercing member unlock mechanism is operable to unlock a lock mechanism by displacing/removing a latch from a valve and/or a plunger and/pre a piercing member.

In one or more embodiments, a portion of the valve and/or plunger and/or piercing member unlocking mechanism that engages/couples with the valve and/or plunger and/or piercing member locking mechanism is disposed in a wall of the vessel, inside the vessel, on an inner surface of the vessel or on an outer surface of the vessel, and combinations thereof.

In one or more embodiments, a portion of the valve and/or plunger and/or piercing member locking mechanism that engages/couples with the valve and/or plunger and/or piercing member unlocking mechanism is disposed in a wall of the vessel, inside the vessel, on an inner surface of the vessel or on an outer surface of the vessel.

In one or more embodiments, any of the valve locking mechanisms, piercing member locking mechanisms, and/or plunger locking mechanisms is selected from a stopper, a latch, a dent, a hole, a channel, a slot, a pin, a retention member, a fastener, and combinations thereof.

In one or more embodiments, any of the valve unlocking mechanisms, piercing member unlocking mechanisms, and/or plunger unlocking mechanisms is selected from a stopper, a latch, a dent, a hole, a channel, a slot, a pin, a retention member, a fastener, and combinations thereof.

Optionally, the valve, piercing member, and/or plunger includes a spring, optionally disposed in a wall of the vessel, for allowing an energy/force that will open/close the valve and/or piercing member and/or plunger.

Optionally, the valve locking mechanisms, piercing member locking mechanisms, and/or plunger locking mechanisms includes a spring, optionally disposed in a wall of the vessel, for allowing an energy/force that will open the valve and/or piercing member and/or plunger.

The decontamination device of the invention includes a housing having side walls, a top wall and a bottom wall, and a wiping member disposed within the housing. The device configured to be coupled to at least two vessels and includes applicable connecting structures/ports or openings for receiving the vessels. The connecting structures/ports/openings may optionally be formed by vertical walls extending from the top and/or bottom walls, forming an inner lumen via which medical substances may be transferred following engagement between, and decontamination of the vessel surfaces. Other port configurations include ports/openings without any walls. The ports may be flush, or surface mounted to the housing. Optionally, a locking and/or unlocking may be disposed in a port of the device, for example, in an internal position within vertical walls of the port. In one or more embodiments, the decontamination device may have a valve. In one or more embodiments, the decontamination device may have a valve and a valve lock mechanism. In one or more embodiments, the decontamination device has a valve and a valve locking mechanism. In one or more embodiments, the decontamination device has a valve unlocking mechanism.

The wiping member is configured to decontaminate a vessel's surface, optionally by a movement of the wiping member across the housing and surface of the vessel, or by movement of the surface of the vessel within the housing. The wiping member may be moveable or may be static. The wiping member may be made of a plastic material, an elastomeric material, a sponge-like material, and combinations thereof.

In an embodiment of the invention, the wiping member is operable to move across and wipe off any contaminates present on the one or more surfaces of the vessels via an actuator extending through an opening of the housing. The actuator is configured to manipulate movement of the wiping member across the housing. In one or more embodiments, the wiping member is operable to move across and wipe off any contaminants present on the one or more surfaces of the vessels via an actuator extending through an opening in the side wall of the housing. Exemplary actuators include, without limitation, a handle, a tab, a button, a touch button, a lever, a gear, a spring, a mechanical actuator, an electric actuator, an air pressure actuator, and combinations thereof.

In one or more embodiments, the wiping member is operable to move across and wipe off any contaminates present on the one or more surfaces of the vessels without any actuator for mediating the wiping action. In accordance with this embodiment, a user may, for example, apply force onto the housing to thereby forcibly manipulate the wiping member to slide across the housing. In accordance with this embodiment, the housing may be flexible, so the user can manipulate the wiping member from the outside and by applying for example press forces onto the housing. In one or more embodiments, the wiping member is moveable from an initial position which precedes the wiping action to an end position which follows the wiping action of the wiping member.

Optionally, the wiping member is static within the housing and the decontamination of a vessel's surface is effective via movement of the vessel, optionally a portion of the vessel, within the housing and via/through/under the wiping member. In accordance with this embodiment, the system optionally comprises a sliding mechanism positioned on at least one of the first vessel and the second vessel, and/or within and/or on the housing, the sliding mechanism configured to allow traveling there along of at least one of the first vessel and the second vessel, such that at least one of the first vessel and the second vessel is configured to move from a first position to a second position. IN one or more embodiments, the sliding mechanism is a rail mechanism. The wiping member is configured to remove contaminants from a surface of at least one of the first vessel and the second vessel at about the time, optionally at the time, of movement of the medical vessels from the first position to the second position. In one or more embodiments, the medical vessel or medical vessel surface slides along the housing and passes through and/or under and/or over the wiping member under great friction, or under a friction sufficient to wipe off any contaminants from the surface of the vessel, such as dirt, bacteria or ambient air particles.

Various types of sliding mechanisms are contemplated as long as they can be connected or engage a vessel and afford traveling there along and allow decontamination of a surface of the vessel. The sliding mechanism may include a rail. The rail may be optionally made from a plastic or polymetric material, but a metallic material or glass material is also applicable. In one embodiment, the rail is manufactured from a plastic material. In one or more embodiments, the plastic material is rigid. In one or more embodiments the vessel surface is positioned on a surface of the rail. In one or more embodiments, the vessel surface is positioned between the rails of the rail. In one or more embodiments, the vessel surface is positioned between a portion of the rails. In one or more embodiments, the rail may be a circumferential rail. In one or more embodiments, the sliding mechanism may include a hinge/pivot mechanism. In one or more embodiments, the sliding mechanism provides for the travelling a vessel between a plurality of positions, such as from a second position to a third position, from a third position to a fourth position, and from a fourth position to a fifth position. In one or more embodiments, the sliding mechanism allows only unidirectional sliding. Alternatively, the sliding mechanism allows for the vessel to retract/return to a previous position.

In one or more embodiments, the wiping member is manufactured from or comprises a material including, but not limited to an elastic material, a rigid material, a flexible material, an expandable material, a polymer, and combinations thereof. In one or more embodiments, the wiping member is manufactured from or comprises an elastomeric material, such as, but not limited to a rubber material. In one or more embodiments, the wiping member is manufactured from or comprises a sponge or a sponge-like material. In one or more embodiments, the wiping member slides across the surface of the one or more vessel(s) under great friction, or under a friction sufficient to wipe off any contaminants from the surface, such as dirt or bacteria. In one or more embodiments, the vessel's surface slides underneath the wiping member under great friction, or under a friction sufficient to wipe off any contaminants from the surface, such as dirt or bacteria.

In one or more embodiments, the wiping member is covered, at least partially, by a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance may be, but is not limited to, an antibacterial substance, an antiviral substance (e.g., an anti-HIV agent), an anti-tuberculin substance, an anti-fungal, and combinations thereof.

In one or more embodiments, the sterilizing substance may be a bactericidal substance, a virucidal substance, and/or a fungicidal substance. Exemplary sterilizing substances include, without limitation, alcohol, isopropyl alcohol, and hydrogen peroxide. In one or more embodiments, the sterilizing or disinfecting substance may be a fluid or a liquid. In one or more embodiments, the sterilizing or disinfecting substance may be a gas. In one or more embodiments, the gas may be a pressurized gas. In one or more embodiments, the sterilizing substance may be housed within the housing of the decontamination device. In one or more embodiments, at least one of the openings and/or ports of the decontamination device are sealed and/or covered thus preventing the sterilizing substance from escaping from inside the housing of the decontamination device. The seals and/or covers maintain the sterility of the decontamination device until about the time of use of the decontamination device. In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, and combinations thereof. In one or more embodiments, the housing may be transparent. In one or more embodiments, the housing may be opaque.

Various sizes and shapes of the housing are applicable. For example, the housing may have an external most length, width, and/or height of less than about 3 inches. In one or more embodiments, the housing may have an external most length, width, and/or height of less than about 25 millimeters (mm). For example, less than about 20 mm, less than about 15 mm, or less than about 10 mm. The housing internal most length, width, and/or height may be less than about 10 mm. For example, less than about 4 mm, less than about 3 mm, or less than about 2 mm. The housing may include one or more compartments which may optionally be separated or divided by the wiping member or by any other means. In one or more embodiments, the housing includes two compartments, wherein a first compartment is kept sterile and a second compartment is configured to receive a vessel which may be contaminated. In one or more embodiments, the sterile compartment can receive a vessel following decontamination of the surface of the vessel.

In one or more embodiments, the housing includes two compartments wherein a first includes a capping mechanism and the second is configured to receive a vessel. In one or more embodiments, the housing includes three, four, or five compartments. The vessel may move from a first position in which a vessel surface is located within the first compartment to a second position in which the vessel surface is located in the second compartment. Optionally, the housing includes a third compartment and the vessel surface is moveable from the second position to a third position. Optionally, the housing includes a fourth compartment, or a fifth compartment and the vessel surface is moveable from a third position to a fourth position and/or a fifth position.

In one or more embodiments, the housing is conveniently light presenting a weight of no more than about 100 grams (gr). For example, less than about 75 gr, less than about 50 mm gr, less than about 25 gr, less than about 10 gr, or less than about 5 gr. In one or more embodiments, the housing may be transparent allowing visualization of its interior, or opaque.

In one or more embodiments, the housing may be a closed chamber which forms a tight, optionally, airtight connection between the vessels. In one or more embodiments, when the ports or openings are connected to vessels or when the ports are covered by a cover or a seal, the housing may form an interior which is hermetically sealed/isolated from surrounding/environmental air. In one or more embodiments, the cover or the seal is frangible, breakable or rupturable. In one or more embodiments, the cover or the seal is pierceable by a piercing member such as a needle.

In one or more embodiments, the housing may optionally include one or more apertures, optionally covered by a filter, to thereby allow releasing air from the housing when actuating the wiping member. Optionally, the one or more apertures may be covered by a valve, to thereby allow releasing of air or pressure from within the housing when actuating the wiping member. In one or more embodiments, the valve may be a one-way valve, thereby allowing the release of air or pressure from within the housing when actuating the wiping member while preventing ambient or environmental air from entering the housing. In one or more embodiments, the filter or valve may be located in or on a wall of the housing's first, second or third compartment.

In one or more embodiments, the system or devices of the invention is provided when the housing is already connected to one or more medical vessels. In accordance with this embodiment, the one or more medical vessels may be integrally manufactured or assembled with the housing and provided or distributed to consumers as such. In accordance with this embodiment, the system may be configured to allow a connection/locking/engagement to additional one or more vessels.

In one or more embodiments, the system includes two or more housings each containing a wiping member. Optionally, the housings are disposed one above the other, providing a top housing and a bottom housing. In accordance with this embodiment, a wiping member disposed on the top housing is configured to wipe a surface of a vessel connected to the top housing and a wiping member disposed on the bottom housing is configured to wipe a surface of a vessel connected to the bottom housing.

In one or more embodiments, the system includes two or more ports which are disposed one above the other, or which are longitudinally aligned with respect to each other. In accordance with this embodiment, the vessels connected to such ports are positioned aligned allowing a direct and aligned fluid passageway between the vessels. In one or more embodiments, the system includes two or more ports which are shifted from each other. Yet, in accordance with this embodiment, a fluid passageway is formed between vessels which is twisted or curved. In yet one or more embodiments, the housing contains a plurality of ports configured for connecting a plurality of vessels to the housing. The system or device is configured so that actuating the wiping member allows wiping the surfaces of all vessels connected to the housing in one action.

In one or more embodiments, the housing has an engagement mechanism configured or adapted to connect the at least one vessel to the housing. In one or more embodiments, the engagement mechanism may be reversible, allowing to detach the engagement between a vessel and a decontamination device. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive. In one or more embodiments, the herein disclosed invention allows transferring medical substances in a contaminant-free, or in a substantially contaminant-free manner.

The term “lock mechanism is interchangeable with the term “locking mechanism”.

The term “unlock mechanism is interchangeable with the term “unlocking mechanism”.

In one or more embodiments, the herein disclosed invention affords an engagement of vessels in a contaminant-free, or in a substantially contaminant-free manner. In one or more embodiments, the herein disclosed invention affords an engagement of vessels in an air-free, or in a substantially air-free manner.

In one or more embodiments, the herein disclosed invention provides a fluidic passageway or communication between vessels in a contaminant-free, or in a substantially contaminant-free manner. In one or more embodiments, the herein disclosed invention provides a fluidic passageway or communication between vessels in an air-free, or in a substantially air-free manner.

In one or more embodiments, the herein disclosed invention, allows to substantially decrease the chances to introduce contaminants within a medical substance, when preparing or administering medical substances for administration to patients. In one or more embodiments, contaminants may be, but are not limited to, bacteria, viruses, fungus, ambient air particles, and pathogens.

As used herein the term “substances” refers to various types of materials that should be kept sterile. The substances may be liquid, semi-solid, or gas. In one or more embodiments, the substances are “medical substances”. As used herein the term “medical substances” refers to and encompasses any of the various pharmaceutical drugs, fluids, nutritional products and the like. In an embodiment of the invention the term “medical substance” is interchangeable with the terms a “pharmaceutical substance”, “a beneficial substance” and “a medicament”.

As used herein the term “contaminant-free” is interchangeable with the term “sterile”, “disinfected”, and “decontaminated”. The term refers to substances that are free or substantially free of ambient air particles and/or pathogens and/or micro-organisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “vessel” refers to any device utilized for containing, transferring or administering substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel. In an embodiment of the invention, the vessel is utilized for containing or housing a medical substance. Various types of vessels are contemplated. The vessel may be selected, without limitation, from a vial, a syringe, a bag, a chamber, a bottle, a container, a cartridge and the alike. In an embodiment of the invention, the term vessel further encompasses elements that can be used to connect between vessels. In accordance with this embodiment, the vessel may be selected, without limitation, from a connector, a connector having a plurality of openings, a syringe, an infusion line, a tubing, an infusion line, a spike, a syringe, a filter, a port and a manifold. In one or more embodiments, the first container is a bag. In one or more embodiments, the herein system include a first vessel and a second vessel. In one or more embodiments, the first vessel is a cartridge configured to fill the second vessel. In accordance with this embodiment, the cartridge includes a mechanism for allowing the fluid transfer. Suitable fluid transfer mechanisms may include, without limitation a plunger, and a moveable wall. Optionally, the cartridge may have various forms, such as a syringe, a vial, a bottle, a bag, etc. In one or more embodiments, the first vessel is a bottle. In one or more embodiments, the first vessel is a syringe and the second vessel is a bag, or vice versa. In one or more embodiments, the first vessel is a syringe and the second vessel is a bottle, or vice versa. In one or more embodiments, the first vessel is a cartridge with a moveable wall and the second vessel is a bag. In one or more embodiments, the first vessel is a cartridge with a plunger and the second vessel is a bag. In one or more embodiments, the first vessel is a cartridge with a moveable wall and the second vessel is a bottle. In one or more embodiments, the first vessel is a cartridge with a plunger and the second vessel is a bottle. In one or more embodiments, at least one of the first and second containers is a bag. In one or more embodiments, at least one of the first and second vessels is a bottle. In one or more embodiments, the vessel is a cartridge having a plunger and protective side walls.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between two containers, between two bottles, between a syringe and a bag, between a syringe and a container, between a syringe and a vial, between a connector and a bag, between a container and a bag, between a vial and a bag, between a container and an infusion line or between a syringe and a connector. Optionally, one vessel is configured to connect a plurality of vessels. For example, one vessel can connect two, or three, or four or five, or six or more vessels. Optionally, a second vessel can connect a plurality of first vessels. Optionally, the second vessel includes a plurality of cavities, each configured to accommodate and/or connect a vessel. In an embodiment of the invention, the herein disclosed devices and systems are disposable. In an embodiment of the invention, the herein disclosed devices and systems are non-disposable.

In one or more embodiments of the invention, the decontamination device has a valve, a valve locking mechanism, a valve unlocking mechanism, and combinations thereof. In one or more embodiments of the invention, the decontamination device has a valve, a valve lock mechanism, and a plunger unlock mechanism. In one or more embodiments of the invention, the decontamination device has a valve unlock mechanism and a plunger unlock mechanism. In one or more embodiments of the invention, the decontamination device has a valve unlock mechanism and a piercing member unlock mechanism. In one or more embodiments, the piercing member unlock mechanism is a needle unlock mechanism. In one or more embodiments of the invention, the decontamination device has a valve unlock mechanism, a plunger unlock mechanism, and a piercing member unlock mechanism.

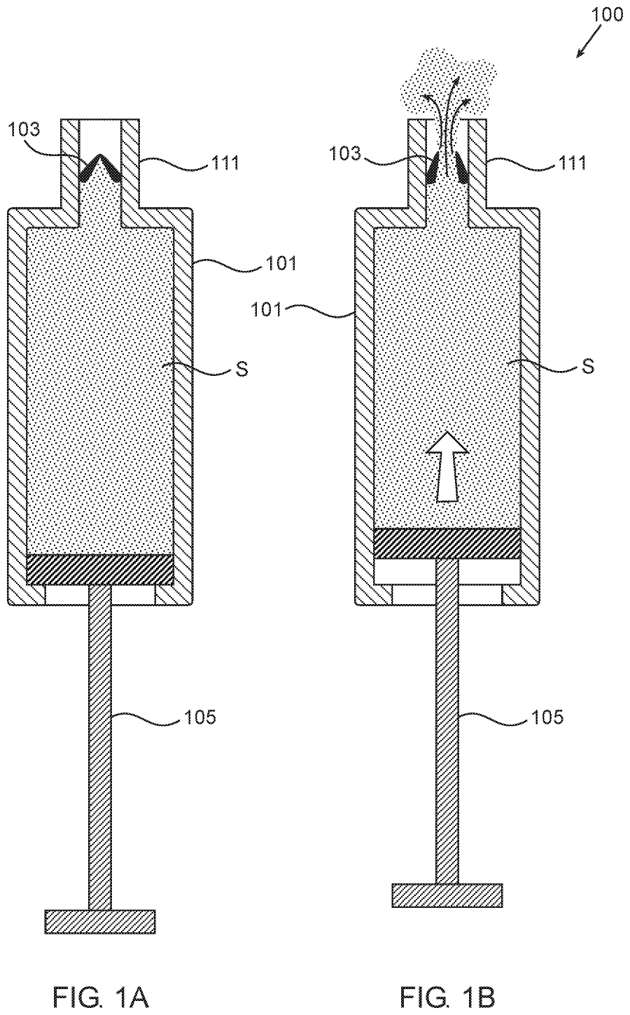

Referring now to the drawings, FIGS. 1A-1B illustrate a syringe 101 having a valve 103 disposed inside the syringe 101. Specifically, valve 103 is disposed within a lumen of tip 111, optionally in the middle of tip 111. At FIG. 1A, valve 103 is shown in a closed configuration thus not allowing a fluid substance “S” to flow into and/or out of the syringe 101. FIG. 1B shows syringe 101 when the valve 103 is in an opened configuration (open valve) which allows substance “S” to flow into and/or out of the syringe 101. Other locations of the valve 103 may be applicable and are herein contemplated. Exemplary locations of the valve 103 are illustrated in the following figures.

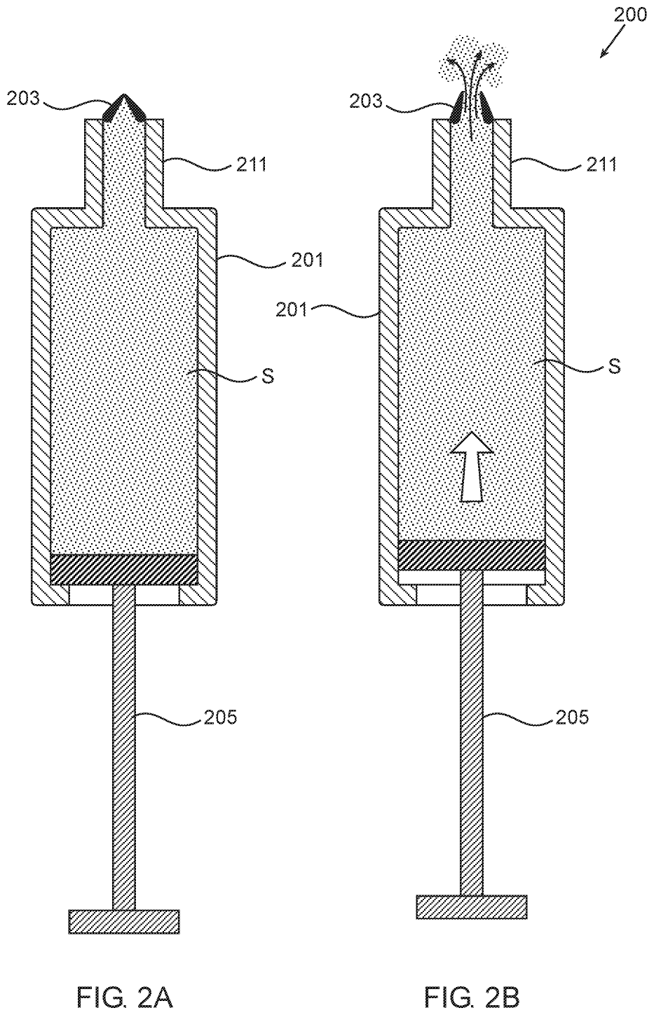

As shown in FIGS. 2A-2B, a valve 203 can be located about the surface of tip 211 of syringe 201. FIG. 2A shows the valve 203 in a closed position when disposed on the surface of tip 211 of syringe 201. The valve 203 of FIG. 2A is in a closed position thus not allowing fluid transfer into and/or out of the syringe 201. FIG. 2B shows the valve 203 in an opened position where the fluid “S” is permitted/allowed to flow into and/or out of the syringe 201. It is to be understood that other types of vessels are herein contemplated and applicable. Non limiting examples of vessels include a container, a bag, a bottle, a vial, a cartridge, a cartridge with protective side walls, a cartridge with circumferential protective side walls, etc.

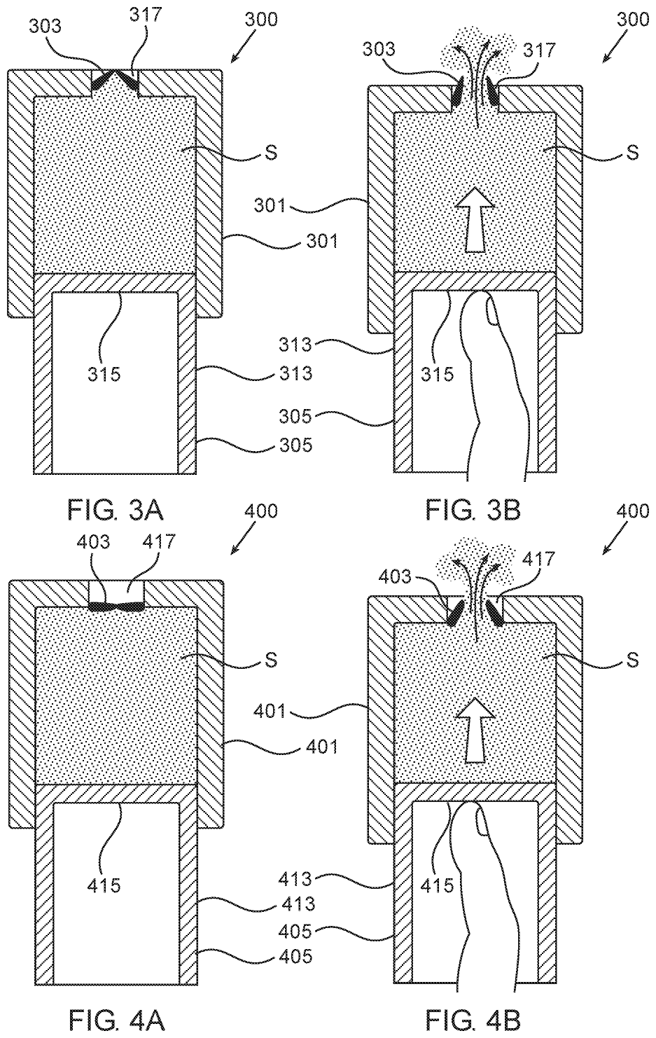

The following FIGS. 3-5 depict exemplary cartridges having a plunger with protective side walls, and a valve. FIGS. 3A-3B illustrate cartridge 301 for accommodating a fluid substance “S”. Cartridge 301 includes a plunger 305 for expelling substance “S” out of the cartridge 301. Plunger 305 has an upper wall 315 and circumferential protective side walls 313. This structure affords protection of a user's finger/hand from contacting any remnants of the substance “S” that may be disposed on the interior walls of the cartridge 301 after the plunger 305 has been pushed upwards towards the valve 303. A valve 303 is disposed in an aperture 317 and coupled from within to the side walls of aperture 317, in a middle location. FIG. 3A illustrates the valve 303 being in a closed position thus not allowing the passage of fluid into and/or out of the cartridge. FIG. 3B shows the valve in an open position which allows a fluidic passageway into and/or out of the cartridge 301.

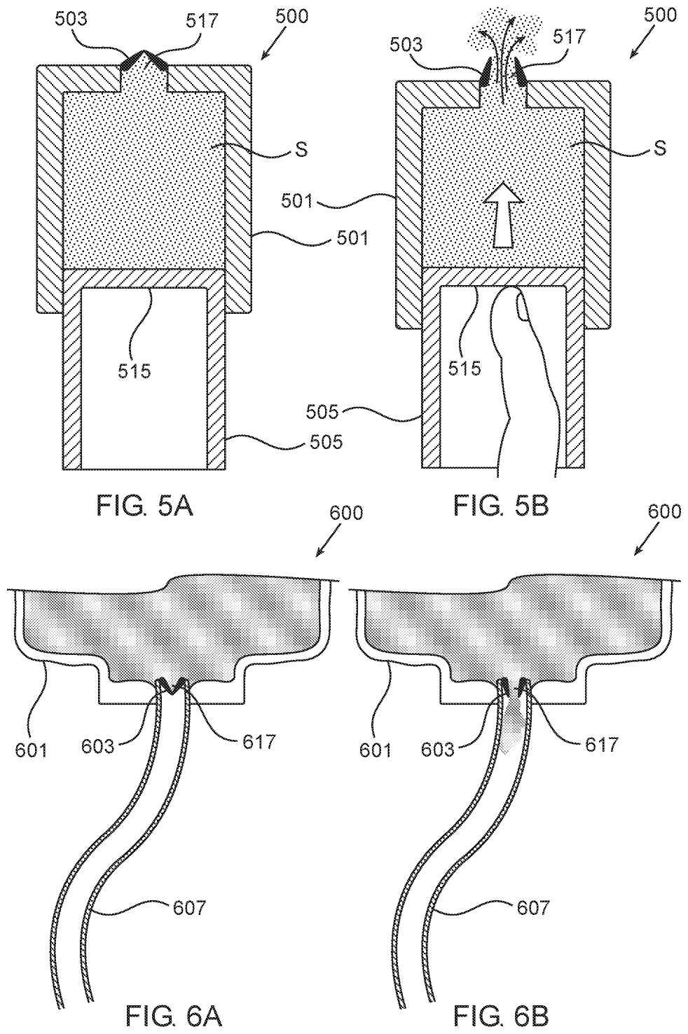

FIGS. 4A-4B illustrate cartridge 401 for accommodating a fluid substance “S”. Cartridge 401 is similar to cartridge 301 with the exception that the valve 403 is disposed about an inner surface of the side walls of aperture 417. Cartridge 401 includes a plunger 405 with upper wall 415 and circumferential protective side walls 413. In FIGS. 5A-5B, the valve 503 is positioned on an exterior surface, and/or about an outer surface of the side walls of aperture 517.

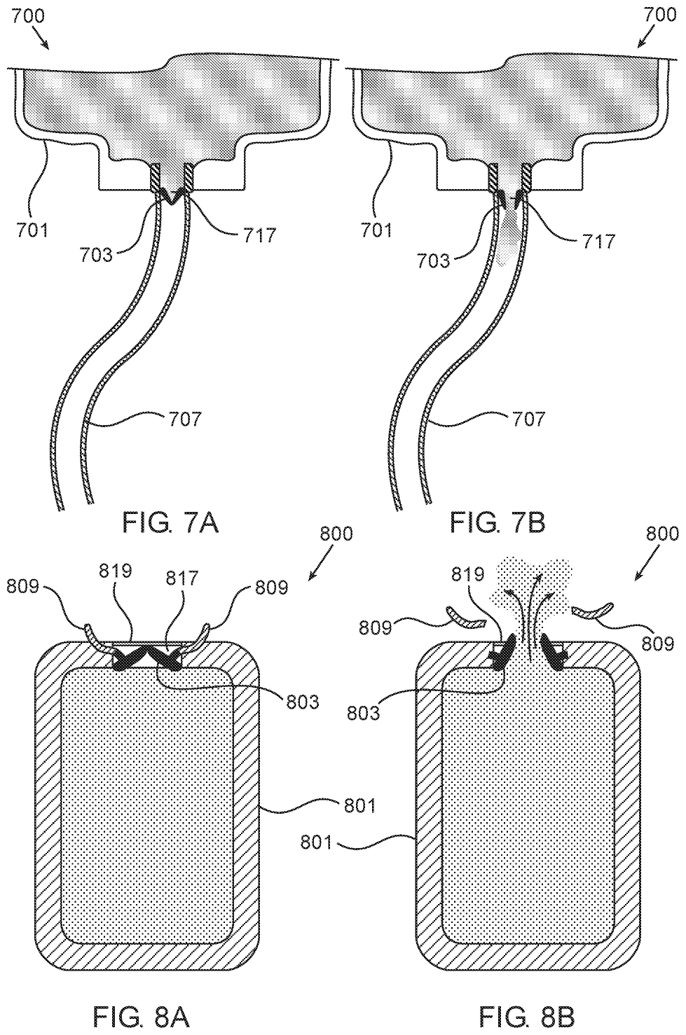

The following FIGS. 6-7 depict exemplary bags attached to an infusion line and having a valve for fluid flow monitoring. In FIGS. 6A-6B, the valve 603 is positioned in an interior position of bag aperture 617. FIG. 6A shows the valve 603 in a closed position. FIG. 3B shows the valve 603 in an open position allowing fluid to pass into and/or out of the bag through the infusion line 607. In FIGS. 7A-7B, the valve 703 is positioned in an exterior position of bag aperture 717. FIG. 7A shows the valve 703 in a closed position. FIG. 7B shows the valve 703 in an open position allowing fluid to pass into and/or out of the bag through the infusion line 707.

The following FIGS. 8-11 depict exemplary cartridges having a valve and a valve lock mechanism. FIG. 8A-8B illustrate cartridge 801 having a valve 803 and a valve locking mechanism having a pole-like form 809. The valve 803 is kept closed and/or can be opened via unlocking of lock mechanism 809. The lock mechanism 809 disposed such that a first end thereof is engaged/coupled/connected to the valve 803 and a second end thereof is exposed to an exterior of the cartridge 801. In accordance with this embodiment, the valve lock mechanism 809 may be disposed and extend through a wall of the cartridge 801. FIG. 8A shows the valve 803 in a closed position wherein fluid is not allowed to move into and/or out of the interior of the cartridge 801. FIG. 8B illustrates the valve 803 is in an open position. The valve locking mechanism 809 has been unlocked externally by a user, thus allowing the valve 803 to become open. The unlocking of the valve locking mechanism may be performed/mediated via various mechanisms. The valve locking mechanism 809 may serve as a stopper which prevents opening the valve 803. Once the valve locking mechanism is displaced/removed from an initial locked position, the valve 809 is free to open. Cartridge 801 includes an opening 817 which is covered by a rubber bung 819 that can be pierced via a needle. Optionally, or alternately, rubber bung 819 may be a seal such as a frangible seal and/or a film.

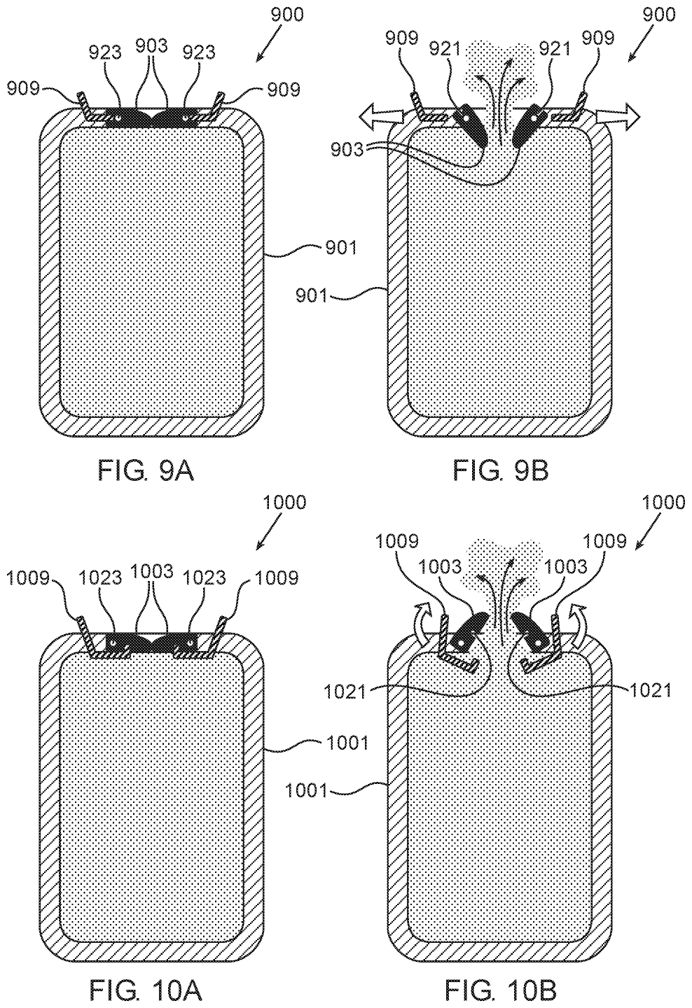

FIGS. 9A-9B illustrate an alternative form/mechanism/configuration of the valve locking mechanism. The lock mechanism herein includes pins 909 attached to valve 903 via dedicated dents 921, wherein an internal end of the valve locking mechanism 909 fits into dent 921 in the valve 903. Once the valve locking mechanism is removed/displaced from dent 921, the valve 903 is free to move. FIG. 9A shows the internal end of the valve locking mechanism 909 disposed in dent 921, thus preventing the valve 903 from opening. FIG. 9B shows the valve lock mechanism when unlocked, i.e., the valve lock mechanism 909 has been withdrawn/removed/displaced from dent 921, allowing the valve 903 to move and open thus allowing fluid to move into and/or out of cartridge 901. Valve 903 may be disposed/connected to a hinge mechanism/hinge 923 attached to cartridge 901 which allows the axial movement of valve 903 from a closed position to an open position.

FIGS. 10A-10B illustrate cartridge 1001 having valve 1003 with dents 1021 and a lock mechanism 1009 having a hook-like form. FIG. 10A shows the valve lock mechanism 1009 wherein an internal end thereof fits into the 1021 of valve 1003 thus locking the valves and preventing opening thereof. An external end of the valve lock mechanism 1009 is disposed on an exterior of the cartridge 1001. FIG. 10A shows an unlocked valve lock mechanism wherein the unlocking is actuated/initiated from an exterior portion of the valve lock mechanism which actuates movement of the valve lock mechanism from a locked initial position to an unlocked final position wherein the interior portion of the valve lock mechanism is displaced/removed from within the dent of the valve as shown in FIG. 10B. The valve is now free to open as shown in FIG. 10B to allow for a fluid to flow into and/or out of the vessel.

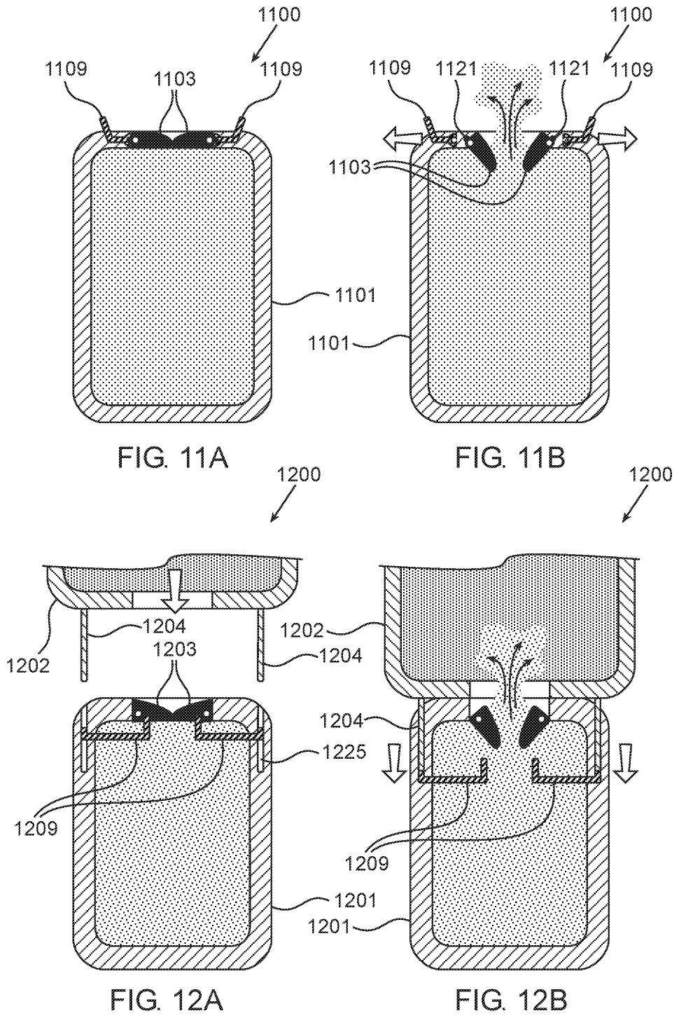

FIG. 11A-11B illustrate cartridge 1101 having a valve 1103 and a lock mechanism in the form of a latches 1109. The latches 1109 hold/fasten the valve 1103 in a locked position via protrusions 1121 within valve 1103. Internal ends of the latches are disposed inside cartridge 1101, optionally within/through a wall of the cartridge 1101. External ends of latches are disposed on an exterior portion. FIG. 11B shows latches 1109 detached from the protrusions 1121 of the valve 1103. The valve 1103 is unlocked and free to move and open. The unlocking of the latches 1109 may be actuated/initiated by applying a force/movement onto the exterior ends of the latches 1109, enabling displacement of the interior ends of the latches 1109 from the protrusions 1121 of the valve 1103. The unlocking of the latches 1109 may be mediated via a push motion, a twist motion, a push and twist motion, and/or a twist and push motion, and combinations thereof. As will be presented in the following figures, the exterior ends of latches 1109 may be configured/adapted to engage/couple with a second vessel and/or with an unlocking mechanism thereof.

FIGS. 12A-12B illustrate a system 1200 which includes a first vessel, here cartridge 1201, and a second vessel 1202. First vessel 1201 includes a valve 1203 and a valve lock mechanism in the form of hooks 1209. Second vessel 1202 includes a valve unlock mechanism which in this case includes two elongated arms 1204 extending externally from a bottom surface of the vessel 1202. The first vessel 1201 and/or second vessel 1202 may accommodate a beneficial substance which may be a medicament, nutritional substance, a diagnostic substance, a diluent, a sterile fluid, etc. Vessels 1201 and 1202 are configured to engage with each other and thereby to establish a fluid communication therebetween. The valve unlock mechanism 1204 is configured to engage with the valve lock mechanism 1209 and thereby to unlock the lock mechanism and allow the movement of valve 1203. Vessel 1201 includes longitudinal channels 1225 at opposing walls thereof. Valve lock mechanism 1209 includes a first end connected to valve 1203 and a second end disposed in the side wall of vessel 1201 and extending into channel 1225 such that the engagement between vessel 1202 and vessel 1201 includes insertion of unlock mechanisms 1204 into channels 1225 and push of lock mechanisms 1209 downwardly. The displacement of lock mechanism 1209 from attachment with valve 1203 will enable unlocking of lock mechanism 1209. The unlocking of the valve lock mechanism 1209 by the valve unlock mechanism 1204 will open the valve thus allowing a fluid to pass into and/or out of the vessel 1201. FIG. 12A shows vessels 1201 and 1202 when spaced apart from each other. Valve lock mechanism 1209 is kept locked and the valve 1203 closed thus preventing a fluid from entering and/or passing out of the vessel. FIG. 12B shows first vessel 1201 engaged/coupled to second vessel 1202. The coupling of the syringe to the vessel may be via a thread, a luer, a luer-lock, a luer-slip, a ratchet teeth mechanism, and/or any other type of engagement mechanism (not shown). At about the time, at the time, and/or after engagement/coupling of the vessels 1201 and 1202, the valve unlock mechanisms 1204 engage/couple with the valve lock mechanisms 1209. The engagement/coupling of valve unlock mechanisms to the valve lock mechanisms unlocks the valve lock mechanisms thus opening the valve 1203 which results in the establishment fluidic communication between the vessels. The opening of the valve lock mechanism 1209 of vessel 1201 may be mediated by a push motion, twist motion, push and twist motion, or a twist and push motion of the second vessel's 1202 valve unlock mechanism(s) 1204 relative to the first vessel's valve lock mechanism(s), or vice versa. The unlocking of the valve lock mechanism 1209 may include a displacement, as herein shown, but can optionally include removal of the valve lock mechanism from the vessel itself.

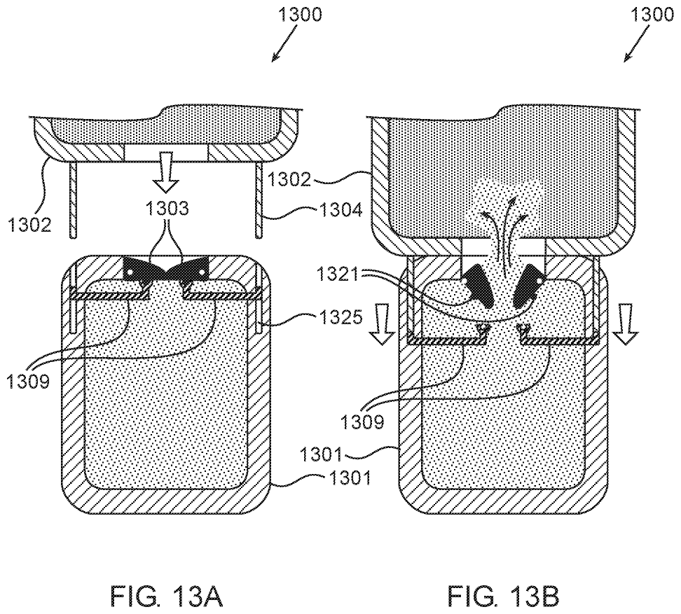

FIGS. 13A-13B illustrate system 1300 which similarly to system 1200 includes a first vessel 1301, a second vessel 1302, a valve 1303, a valve lock mechanism 1309 and a valve unlock mechanism 1304 and channels 1325. The valve lock mechanism 1309 in this case has latch-like form at first ends thereof which hold/fasten the valve 1303 in a locked position via protrusions 1321. Second ends of latches 1309 disposed within the side walls of cartridge 1301 such that the engagement between first vessel 1301 and second vessel 1302 allows to unlock lock mechanism 1309 when elongated arms 1304 enter channels 1325 and thereby push downwardly second ends of latches 1309. The valve 1303 can now open and allow the fluid transfer between vessel 1301 and vessel 1302.

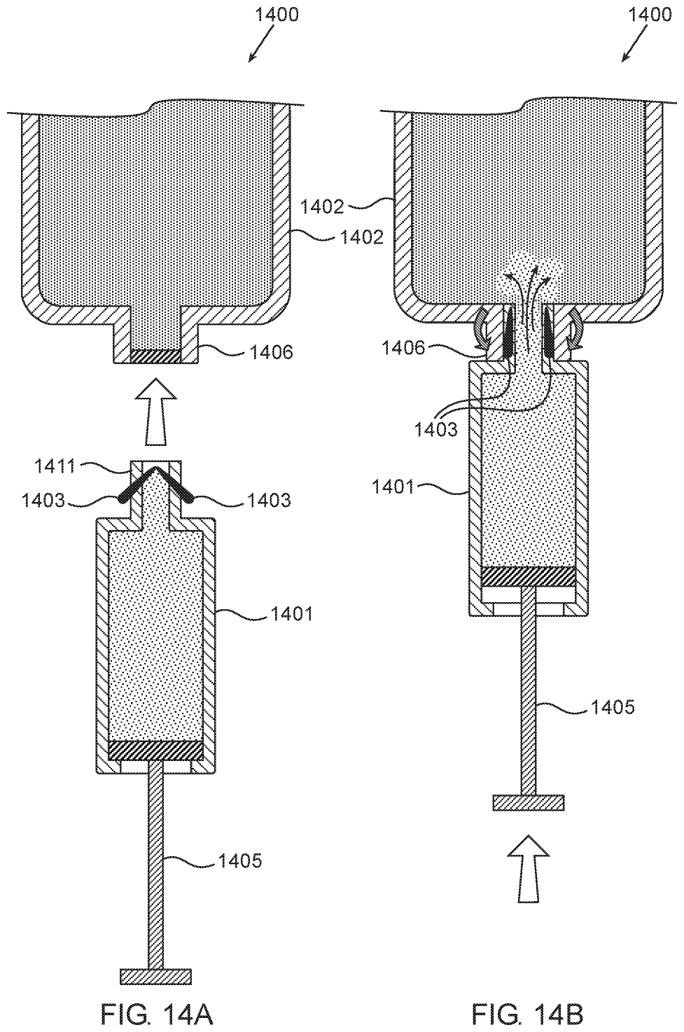

FIGS. 14A-14B illustrate yet another system 1400 having a first vessel 1401, a second vessel 1402 and a valve 1403 disposed in first vessel 1401, on tip 1411 thereof. Valve 1403 disposed such to extend from an internal position within tip 1411, via the side walls, to an external position in tip 1411. The protruding portions of valve 1403 may serve to mediate the unlocking of the valve 1403 when first vessel 1401 engages second vessel 1402. Since second vessel 1402 includes a port 1406 with side walls that can connect tip 1411 in a manner that allows pushing the externally protruding portions of valve 1403, opening of the valve 1403 occurs. Optionally, as demonstrated herein in FIG. 14B the valve 1403, or at least a portion thereof can be incorporated in the side walls of vessel 1401.

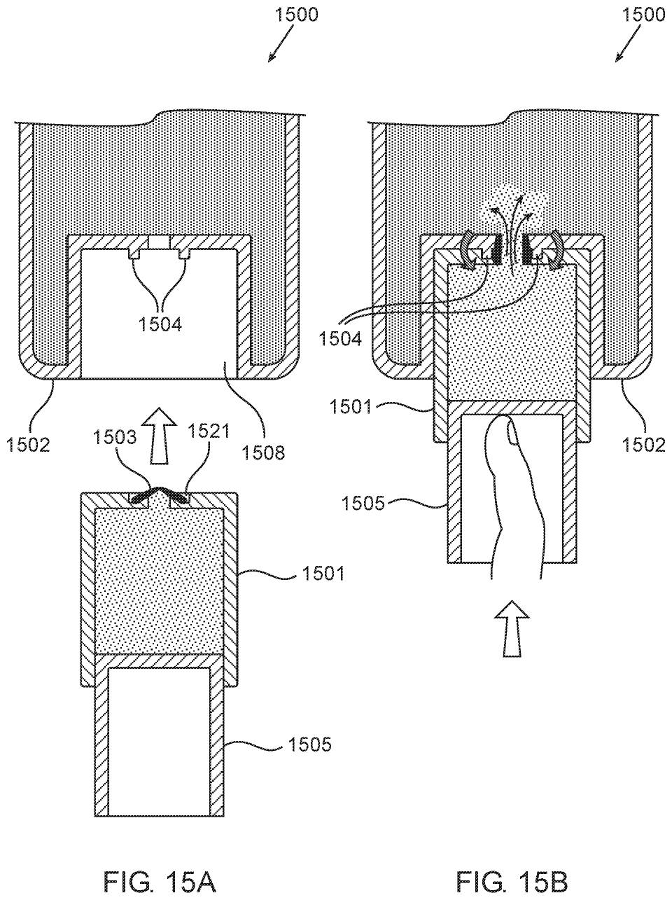

In FIGS. 15A-15B cartridge 1501 includes a valve 1503 and second vessel 1502 includes a valve unlock mechanism in the form of tenons 1504 that can enter sockets 1521. The valve 1503 includes second ends that reside in sockets 1521 such that the engagement between vessel 1501 and vessel 1502 includes an attachment between tenons 1504 and sockets 1521 and opening of the valve 1503 (FIG. 15B). Second vessel 1502 includes a cavity 1508 having a size and shape that allows accommodating therein at least a portion of vessel 1501. FIG. 15A shows the vessels 1501 and 1502 when spaced apart and FIG. 15B shows the vessels upon an attachment thereof and when a user pushes plunger 1505 upwardly to allow fluid flow from first vessel 1501 to second vessel 1502.

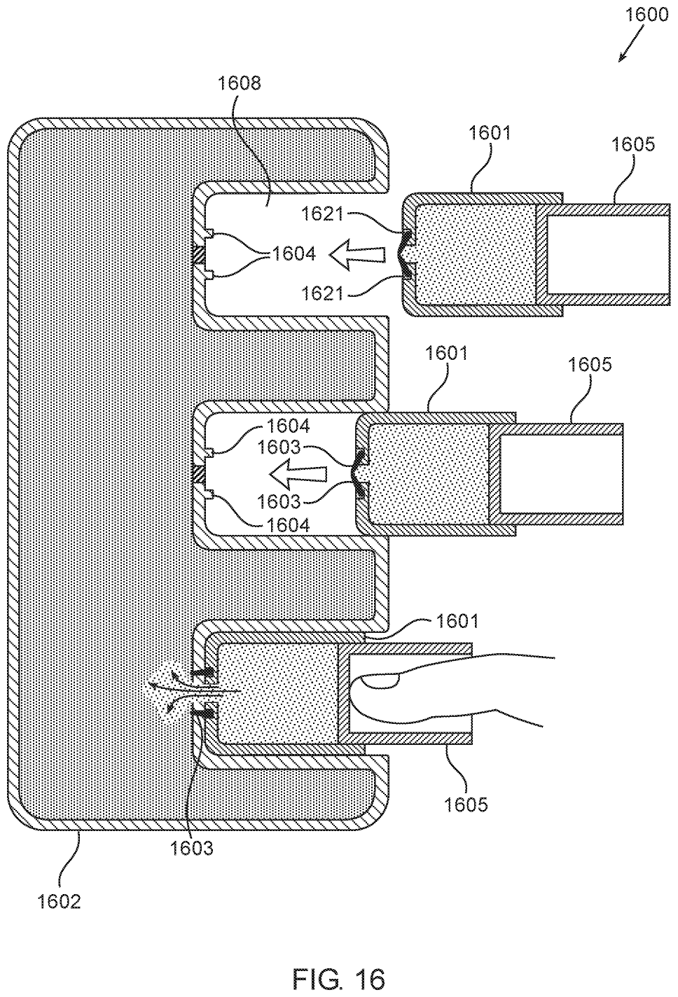

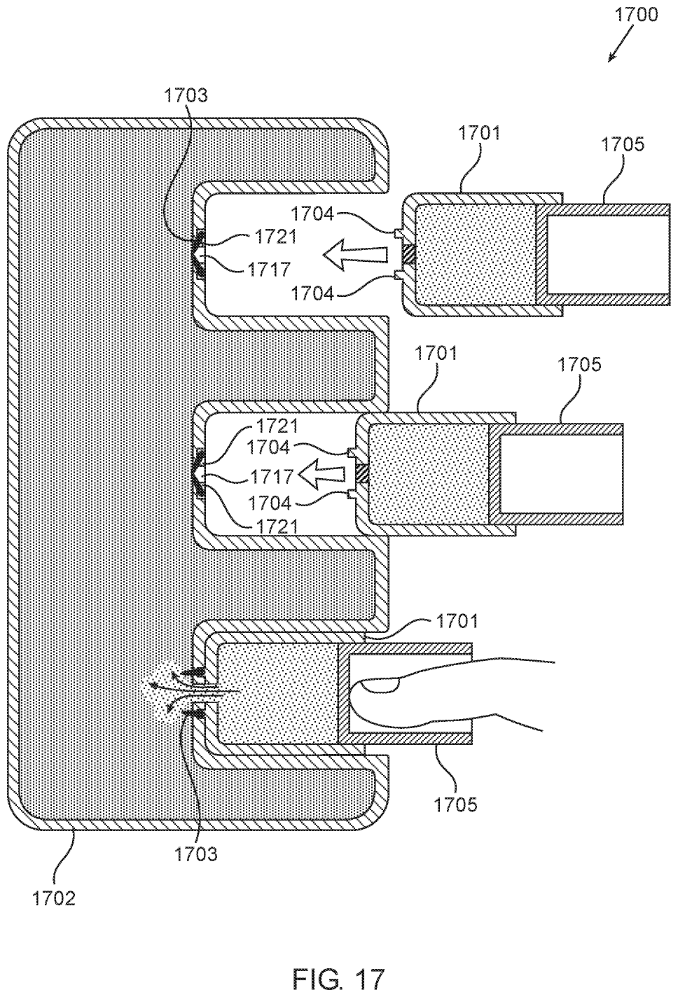

The following FIGS. 16 and 17 illustrate systems 1600 and 1700 with a plurality of first vessels and a second vessel that can be coupled thereto. Said systems constructed such that the plurality of first vessels feed the second vessel, or vice versa. In FIG. 16, a second vessel in the form of bag 1602 includes a plurality of cavities 1608 for accommodating therein first vessels 1601. First vessel in the form of a cartridge 1601 having a plunger 1605 includes a valve 1603. The cavities 1608 having valve unlocking mechanisms in the form of tenons 1604 located inside the cavities of the bag and protrude externally from a side wall in the cavities 1608. The valve unlocking mechanisms 1604 may be disposed on a surface of the cavities, on a wall of the cavities, and/or in various alternative positions on the bag 1602. The valve unlocking mechanisms 1604 are configured in size and/or shape to engage and/or couple with sockets 1621 disposed on a surface of the vessel(s) 1601. It is to be noted that alternative forms of first and/or second vessels are applicable and herein contemplated. For example, first and/or second vessels may be a bottle, a manifold, a connector, syringes, and any of the alike.

FIG. 17 shows system 1700 which is similar to system 1600 but valve 1703 disposed on apertures 1717 of second vessel 1702 and the unlock mechanism with tenons 1704 disposed on first vessel 1701. Sockets 1721 accommodate therein a first end of the valve 1703 such that the engagement between tenons 1704 and sockets 1721 allows the opening of valves 1703.

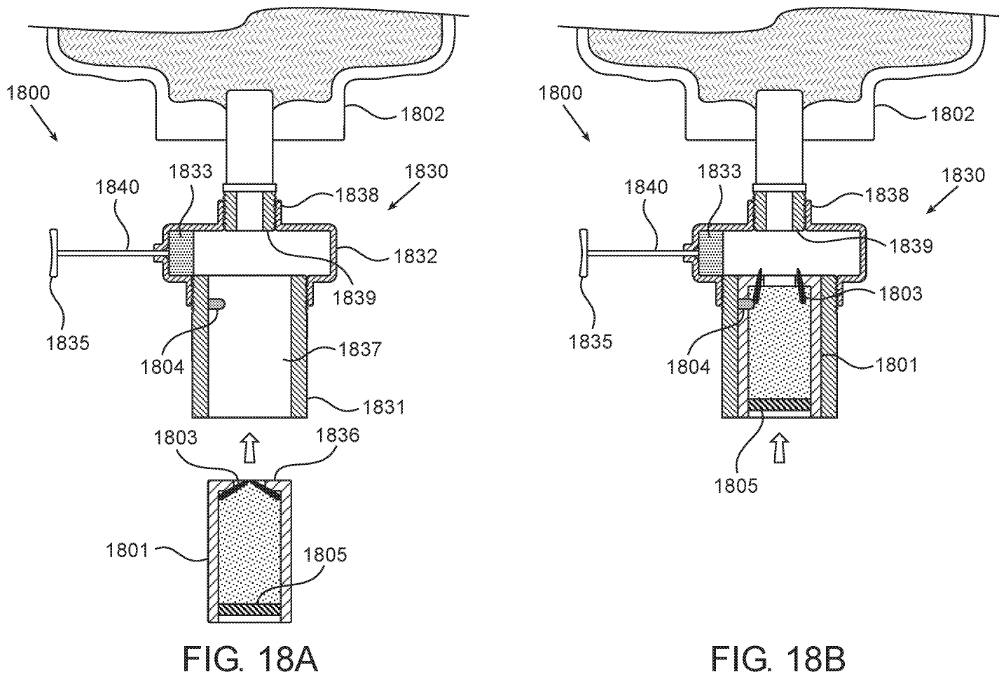

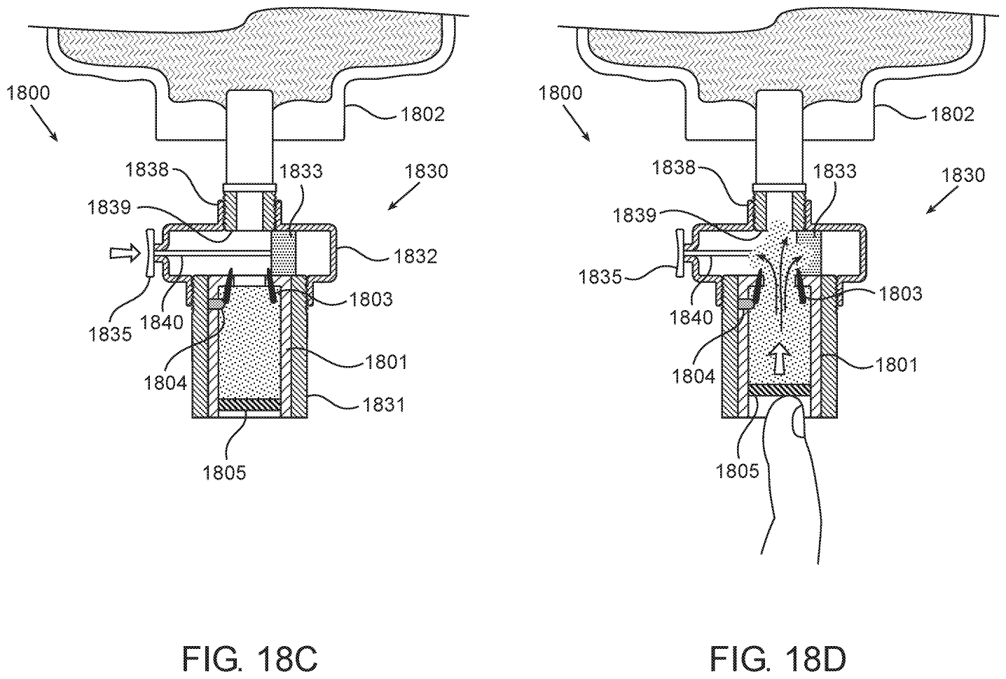

The FIGS. 18A-18D illustrate system 1800 for fluid transfer between vessels 1801 and 1802 that includes a decontamination device 1830 having a housing 1832 configured to decontaminate one or more of the surfaces 1839 and/or 1836 of vessels 1802 and 1801. First vessel 1801 is having a cartridge-like form and second vessel has a bag-like form, nevertheless alternative forms of vessels are contemplated and applicable. For example, vessel 1801 may be a syringe, vial, bottle, bag, container, etc. In another example, vessel 1802 may be a bottle, container, connector, manifold, etc. In system 1800, the connection between vessels 1801 and 1802 is via the decontamination device 1830. Decontamination device 1830 includes a first port 1831 which encloses a lumen 1837 for accommodating therein first vessel 1801 or a portion thereof. Decontamination device 1830 further includes a second port 1838 for accommodating therein second vessel 1802 or a portion thereof. The decontamination device 1830 has a moveable wiping member 1833 configured to move across the device 1830 and to thereby decontaminate surfaces 1836 and/or 1839. Wiping member 1833 is manipulated externally to move across the device 1830 via a wiping member actuator having a form of an actuator/handle 1835 with a rod 1840. Rod 1840 positioned through a wall of the device 1830 and connects handle 1835 to the wiper 1833. When the handle 1835 is pushed to the right and/or slides across the decontamination device 1830 by a user, the user's force transfers the energy placed on the handle 1835 through the rod 1840 to the wiper 1833 and moves the wiper across the decontamination device 1830. In system 1800, a valve 1803 disposed on cartridge 1801 and an unlocking mechanism 1804 disposed on port 1831. The valve unlock mechanism 1804 is shown disposed on an inner surface of a wall of port 1831 of the decontamination device 1832. However, the valve unlock mechanism may be disposed anywhere on/in the decontamination device 1832. The valve unlock mechanism may be disposed on a surface of the decontamination device, on an exterior surface of the decontamination device, on an interior surface of the decontamination device, extending through a wall of the decontamination device, located inside a wall of the decontamination device, and/or disposed on/in a port of the decontamination device. The unlock mechanism includes a moveable ball which can be retracted and extended. Optionally, vessel(s) 1801 and/or 1802 may be provided when already attached, optionally integrally manufactured with device 1830. At about the time of or following the engagement between first vessel 1801 and decontamination device 1830, valve 1803 is unlocked by the unlock mechanism 1804 (FIG. 18B). Surfaces 1836 and/or 1839 can be decontaminated in parallel, or thereafter the engagement by manipulation of actuator/handle 1835 (FIG. 18C). A fluid transfer may then be performed by a user pushing plunger 1805 towards second vessel 1802. FIG. 18A shows the decontamination device 1830 and second vessel 1802 when connected to each other but spaced apart from cartridge 1801. FIG. 18B shows the cartridge 1801 disposed inside port 1831 of the decontamination device 1830. The cartridge 1801 is engaged/coupled to the decontamination device 1830 and a surface 1836 of the cartridge is disposed inside the decontamination device 1830. The valve unlock mechanism 1804 pushes valve 1803 of the cartridge 1801. The wiper 1833 can now be actuated by a user by pushing/pulling/sliding the handle 1835 across/into the decontamination device thus moving the wiper across the interior of the decontamination device which will wipe off a surface of the cartridge 1801. In FIG. 18C the wiping member 1833 is shown actuated to wipe off surfaces 1836 and also optionally surface 1839, thus decontaminating the vessels 1801 and 1802. Upon and/or after opening of the valve as shown in FIG. 18D, a fluidic communication may be established between the cartridge 1801 and the decontamination device 1830. It is to be noted that any of the lock/unlock mechanisms, and vessels disclosed herein above and in the following figures is applicable to the herein disclosed system 1800.

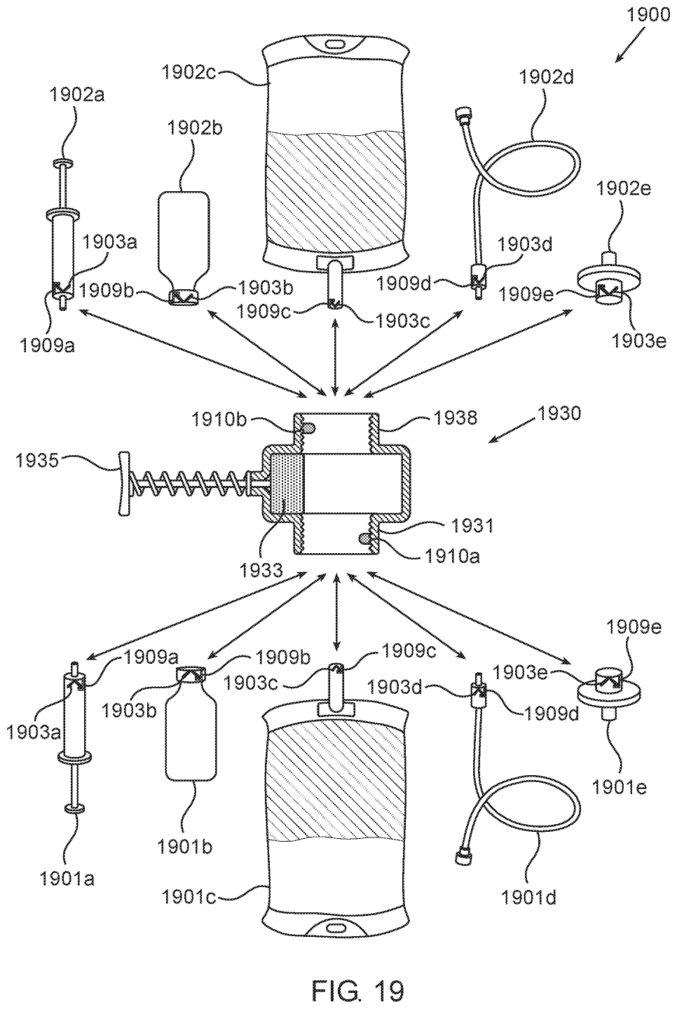

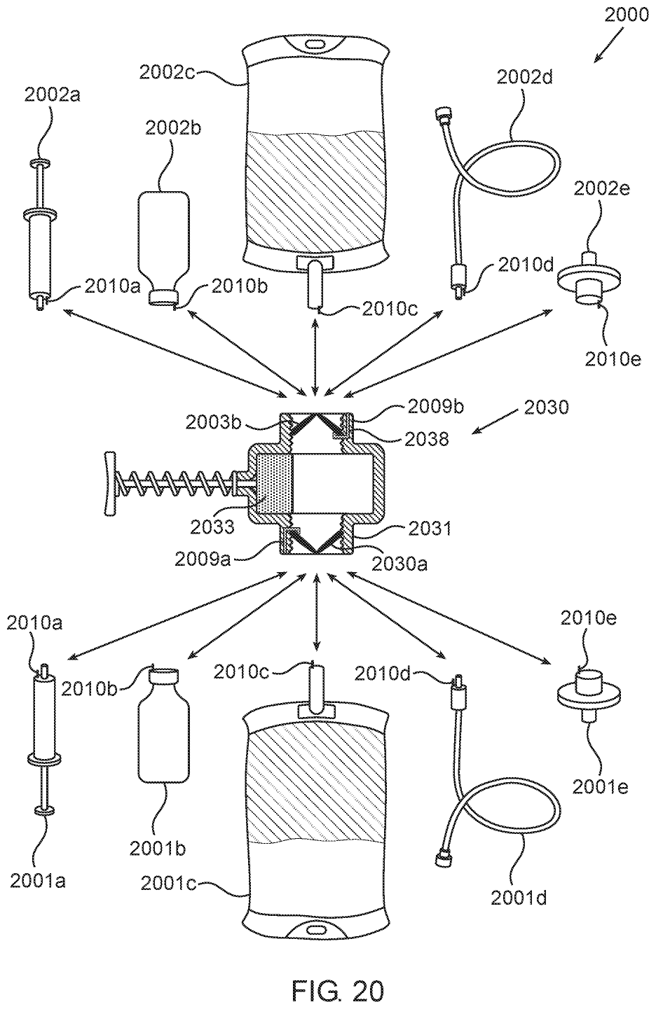

Systems with a decontamination device having a moveable wiping member that can be used as connectors are shown in FIGS. 19-20. The systems 1900-2000 illustrate connectors configured to be coupled to a first and a second vessel and a wiping member that is disposed within the connector and that can wipe off the surface of both vessels. In system 1900 (FIG. 19), the decontamination device 1930 with wiper 1933 is in the form of a connector and has a first port 1931 and a second port 1938 for allowing an engagement with a first vessel and second vessels. Various forms of first vessel are contemplated, such as, syringe 1901a, bottle/vial 1901b, bag 1901c, infusion line 1901d and filter 1901e. Various forms of second vessel are contemplated, such as, syringe 1902a, bottle 1902b, bag 1902c, infusion line 1902d and filter 1902e. The first vessels may include a valve 1903 and a valve lock mechanism in the form of hooks 1909. A valve 1903, with a similar lock mechanism 1909 or any other lock mechanism as herein described, may be disposed on second vessels. The connector 1930 further includes a valve unlocking mechanism in the form of retractable balls 1910a/1910b. The wiper 1933 configured to move across the decontamination device 1930 (from left to right) when handle 1935 is pushed to the right (optionally across the decontamination device). The wiper 1933 will wipe off a surface of first vessel and/or second vessel upon engagement/coupling thereof to the decontamination device 1930.

In FIG. 20, a system 2000 with a connector 2030 and wiper 2033 is shown. Here, the connector includes valves 2030a and 2003b on first port 2031 and on a second port 2038. A lock mechanism in the form of hooks 2009a and 2009b are further incorporated in device 2030 to maintain the closed configuration. Various forms of first vessels are contemplated, such as, syringe 2001a, bottle 2001b, bag 2001c, infusion line 2001d and filter 2001e. Various forms of second vessel are contemplated, such as, syringe 2002a, bottle 2002b, bag 2002c, infusion line 2002d and filter 2002e. A valve unlock mechanism in the form of pins 2010 may be incorporated to the first and/or second vessels. The valve unlock mechanisms 2010a, 2010b, 2010c, 2010d, and 2010e are configured to unlock hooks 2009a and 2009b when the first and/or second vessels engage/couple with/to connector 2030.

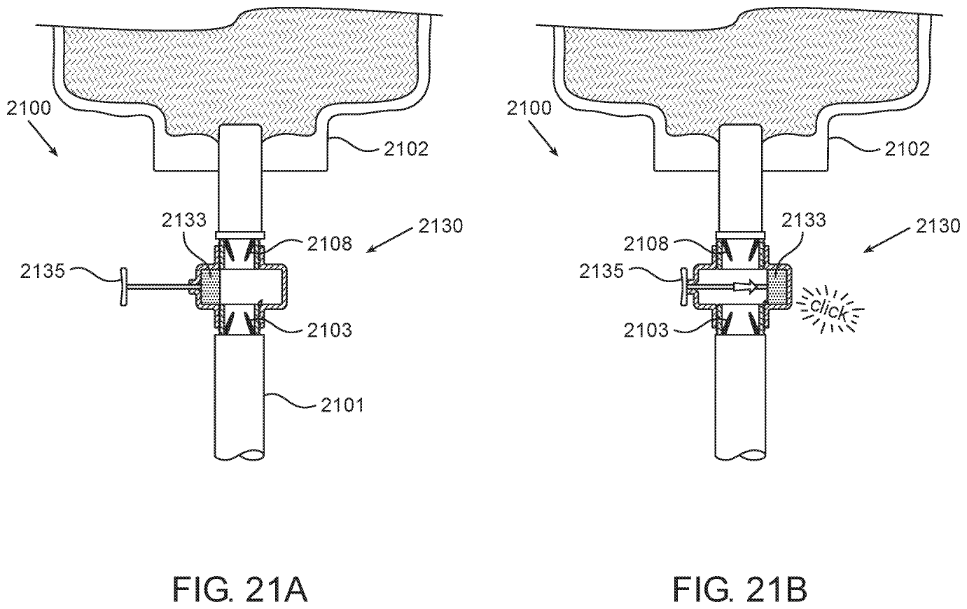

FIGS. 21A-21B illustrate system 2100 with decontamination device 2130 coupled to first vessel 2101 and second vessel 2102. Each of the vessels 2101 and 2102 includes a valve, i.e., valves 2103 and 2108, respectively. Following the engagement between first vessel 2101 and/or second vessel 2102 with decontamination device 2130, a user can manipulate movement of wiping member 2133 via handle 2135 to thereby allow the decontamination of vessels 2101 and/or 2102. Valves 2103 and 2108 are opened at about the time and/or after engagement of first vessel 2101 and second vessel 2102 to the decontamination device 2130. Optionally, or alternately, valves 2103 and 2108 are opened at about the time and/or after coupling of first vessel 2101 and/or second vessel 2102 to the decontamination device 2130. Decontamination device 2130 opens valve 2103 and/or valve 2108.

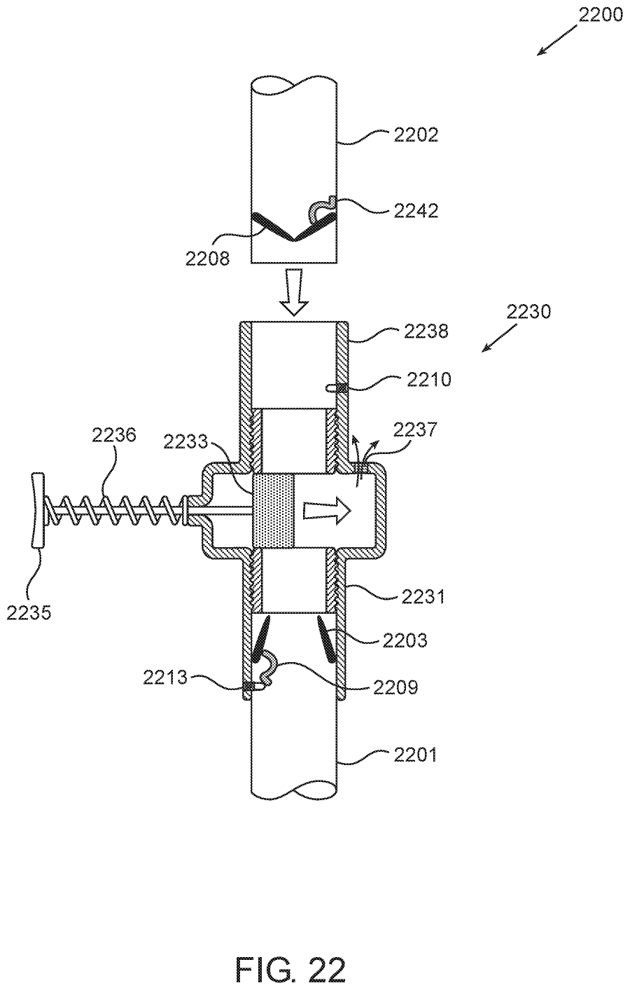

FIG. 22 illustrates yet another exemplary system 2200 which includes a decontamination device 2230 having ports 2231 and 2238 with extending peripheral walls. The extending walls of the decontamination device 2230 are configured in size and/or shape to accommodate (allow entry) of vessels 2201 and 2202. First vessel 2201 is shown disposed inside/within the extending side walls of the decontamination device 2230. Second vessel 2202 is shown prior to an engagement with device 2230. Each of the vessels 2201 and 2202 includes a valve, i.e., valves 2203 and 2208, respectively. The valves 2203 and 2208 maintained locked/closed prior to an engagement with device 2230 via valve lock mechanisms 2209 and 2242. Unlock mechanisms 2213 and 2210 in the form of retractable balls 2209 and 2210 are provided to unlock lock mechanisms 2209 and 2242, respectively. Device 2230 further includes a filter 2237 to allow releasing air from the device when actuating the wiping member 2233. Device 2230 further includes handle 2235 which is spring 2236 loaded to allow the retraction of handle 2235 back into its initial position following the decontamination action.

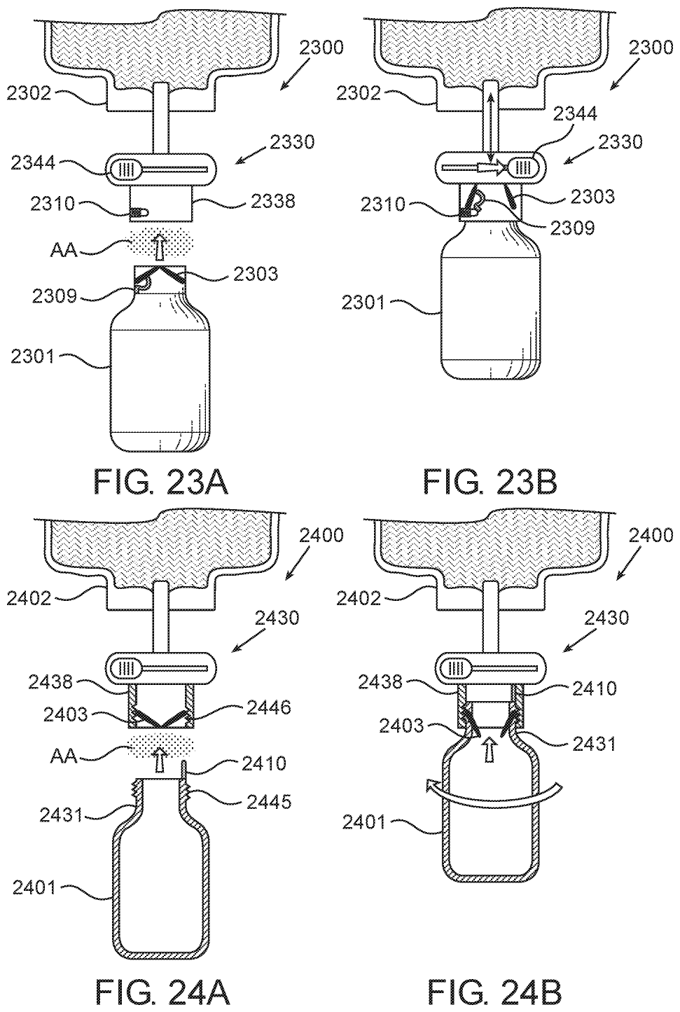

In system 2300 of FIGS. 23A-23B the decontamination device 2330 includes a wiping member (not shown) moveable via movement of tab 2344. The wiping member (not shown) is disposed inside decontamination device 2330. Tab 2344 is disposed on the exterior of the decontamination device and is connected to the wiper (not shown) disposed on the interior of the device 2330. A first vessel 2301 in the form of a vial includes a valve 2303 which is locked by lock mechanism 2309 in the form of a pin. A valve unlock mechanism 2310 is incorporated in port 2338. The valve unlock mechanism 2310 is shown disposed on an interior wall of port 2338, however other locations within/on the decontamination device 2330 can be applicable and are herein incorporated (e.g., about the surface of port 2338). The valve unlock mechanism 2310, in the form of a retractable ball, can be engaged with and/or coupled to valve 2303 and/or to valve lock mechanism 2309 and thereby open the valve 2303. In FIG. 23A, the vessel 2301 is shown prior to an engagement with device 2330. Ambient Air “AA” surrounds the vessel 2301. Following the engagement between vessel 2301 and device 2330, the wiping member can be moved by a user moving the tab 2344 from left to right (FIG. 23B). As the tab 2344 slides across the decontamination device 2330, the wiper moves across the interior of the decontamination device 2330 and wipes off a surface vial 2301 and/or 2302.

FIGS. 24A-24B illustrate system 2400 which is similar system 2300 but valve 2403 disposed within port 2438 of decontamination device 2430. An engagement mechanism in the form of thread 2445 and complementary thread 2446 may be incorporated in port 2431 and port 2438 of vessel 2401 and device 2430, respectively, to allow a safe connection between vessel 2401 and device 2430. The system 2400 further include a valve unlock mechanism 2410 in the form of a pin which mediated the opening of valve 2403 upon the engagement between vessel 2401 and device 2430.

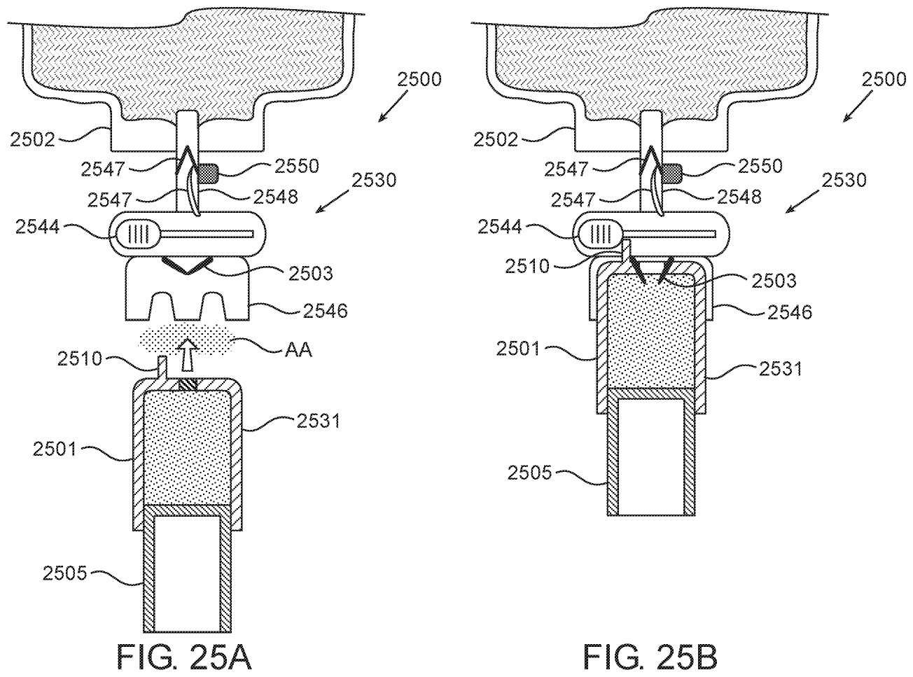

FIGS. 25A-25B illustrate system 2500 having first vessel in the form of cartridge 2501 and second vessel in the form of bag 2502 fluidly connected via decontamination device 2530. Ratchet teeth 2546 engagement mechanism is configured to clasp cartridge 2501 or a port 2531 thereof. Decontamination device 2530 has a valve 2503 disposed in an aperture thereof (not shown). System 2500 also has an unlock mechanism 2510, in the form of a tenon, configured to engage with and/or couple with and/or open valve 2503. Upon opening of valve 2503 a fluidic communication may be established between vessel 2502 and vessel 2501. A piercing member 2547 may be further incorporated in system 2500, optionally, on port 2548 of vessel 2502, to pierce a seal or cover (not shown) of second vessel 2502. A further valve 2547 may be further incorporated to ensure a one-way fluid passageway. The valve 2547 may be actuated upon a user actuating valve actuator 2550. Piercing member 2547 may be a needle, optionally, a hollowed needle, but various types of piercing members are contemplated, such as members that possess a sharp edge. Steps of establishing a fluid communication between vessel 2501 and vessel 2502 include one or more of: insertion of or connection/engagement between the vessels, actuation of the wiping member via tab 2544 thereby effecting a decontamination of one or both vessels, opening valve 2547 by actuating actuator 2550 and transferring a substance by pushing plunger 2505 towards second vessel 2502. The insertion of or connection/engagement between the vessel 2501 and device 2530 opens valve 2503.

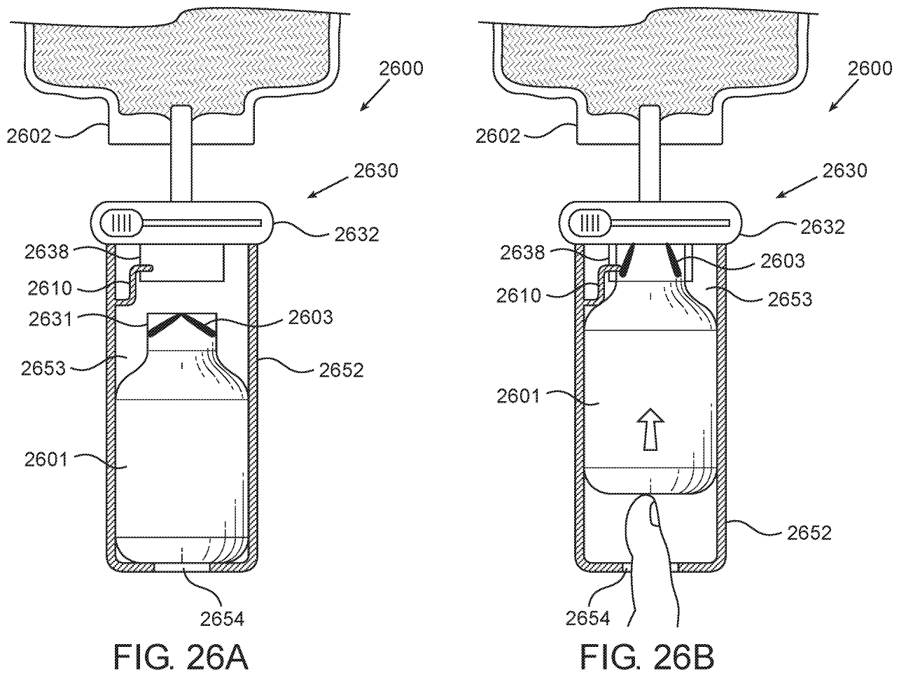

FIGS. 26A-26B illustrate system 2600 having a first vessel 2601, and a second vessel 2602 connected to decontamination device 2630. First vessel 2601 includes a valve 2603. Decontamination device 2630 includes downward extending walls/vertical walls 2652 enclosing a lumen 2653 within which vessel 2601 can be stored. Device 2630 further includes a port 2638 for connection to port 2631 of vessel 2601. An unlock mechanism 2610 extends from downward extending walls 2652 into port 2638 to allow the unlocking of valve 2603. FIG. 26A illustrate vessel 2601 located/disposed within housing 2632 and between downward extending walls 2652 of the decontamination device 2630. In FIG. 26B vessel 2601 is connected to port 2638. Valve 2603 is unlocked by valve unlock mechanism 2610, allowing a fluidic communication between 2601 and 2602 when a user pushes vessel 2601 via aperture 2654 which allows insertion of a user's finger/hand.

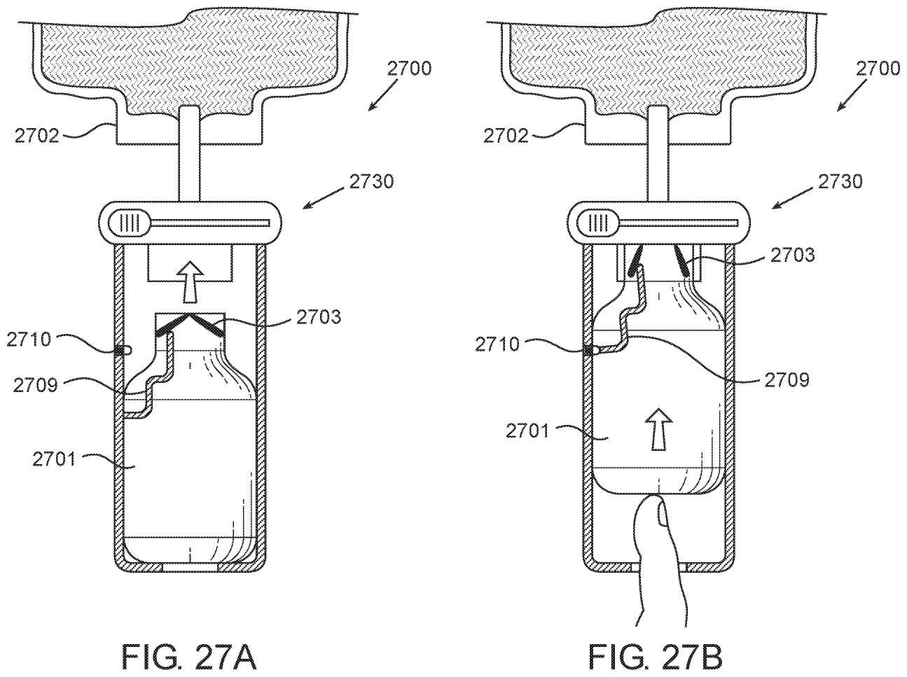

FIGS. 27A-27B illustrate system 2700 having a first vessel 2701, and a second vessel 2702 connected to decontamination device 2730. System 2700 is similar to system 2600 with the exception that vessel 2701 includes a valve lock mechanism 2709 that maintains valve 2703 in a locked position prior to an engagement between vessel 2701 and decontamination device 2730 (FIG. 27A). Upon the engagement between vessel 2701 and device 2730, valve unlock mechanism 2710 unlocks valve lock mechanism 2709, allowing opening of valve 2703.

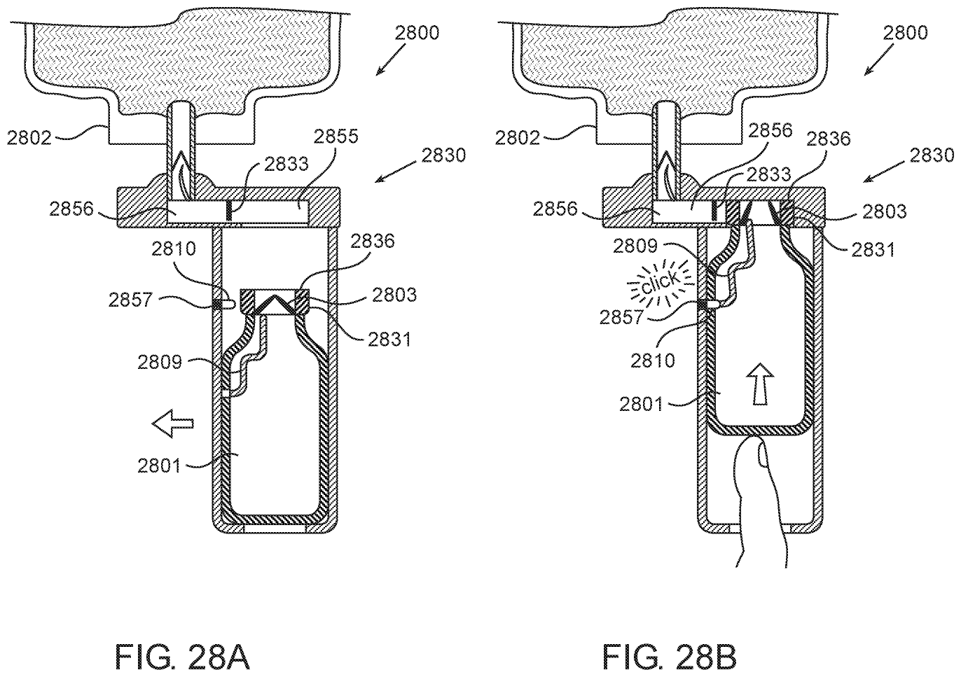

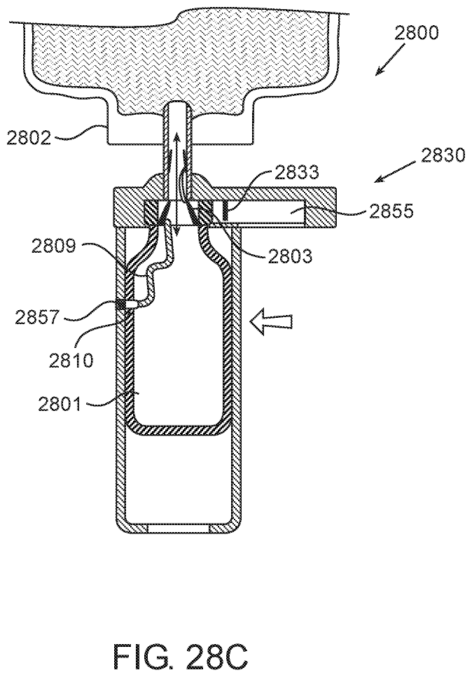

FIGS. 28A-28C illustrate yet another exemplary system 2800 with a decontamination device 2830 having a static wiping member 2833 disposed between a first compartment 2855 and a second compartment 2856 of the device 2830. Vessel 2801 is moveable within the device 2830 between a first position in first compartment 2855 (FIG. 28B) and a second position in second compartment 2856 (FIG. 28C). Wiping member 2833 is configured to decontaminate the surface/seal 2836 upon movement of the vessel 2801 towards second compartment 2856. The system 2800 further includes a valve 2803 disposed within port 2831 of vessel 2801, a valve lock mechanism in the form of curved rod 2809, and a valve unlock mechanism in the form of retractable ball 2810 which may be spring 2857 loaded. The engagement between vessels 2801 and 2802 includes an attachment/contact or proximity between unlock mechanism 2810 and lock mechanism 2809 such that retractable ball 2810 is contracted via spring 2857 when the vessel 2801 engages device 2830 and expands upon positioning of vessel 2801 within first compartment 2855. This enables the unlocking of lock mechanism 2809 and opening of valve 2803.

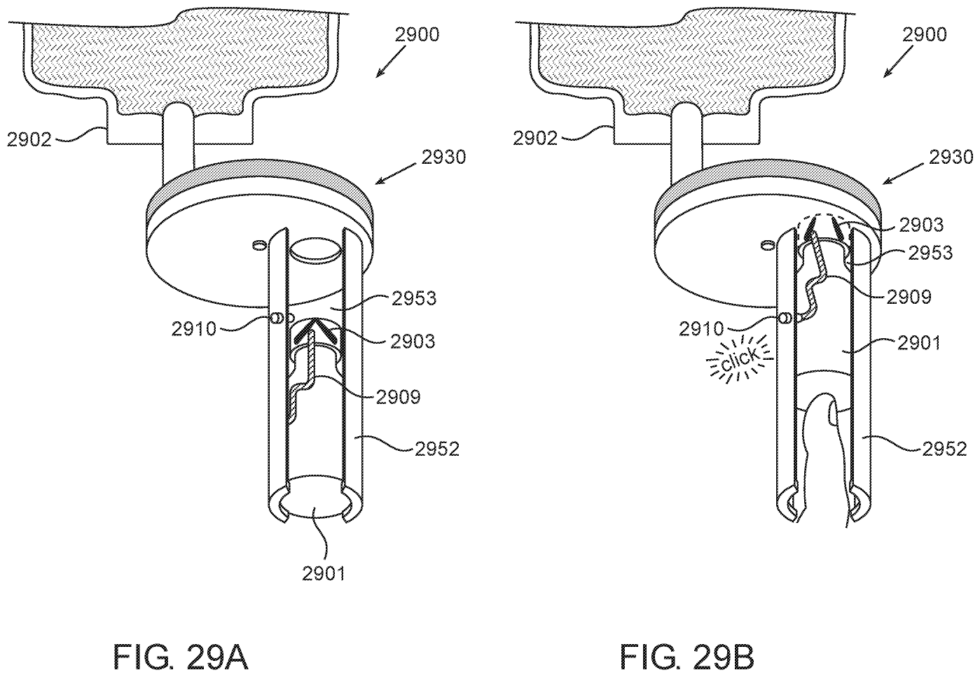

FIGS. 29A-29B illustrate system 2900 which is similar to system 2800 but includes a decontamination device in the form of discs 2930. Device 2930 includes downward extending walls 2952 which enclose a lumen 2953 for accommodating therein vessel 2901. The vessel 2901 and device 2930 configured such that vessel 2901 is moveable within the device via rotational movements. A valve 2903, and a valve lock mechanism 2909 are disposed in vessel 2901. When vessel 2901 engages device 2930 via an aperture thereof, the valve unlock mechanism 2910 effects an opening of valve 2903 via unlocking lock mechanism 2909. A “click” sound may be emitted at the time of engagement between vessel 2901 and device 2930.

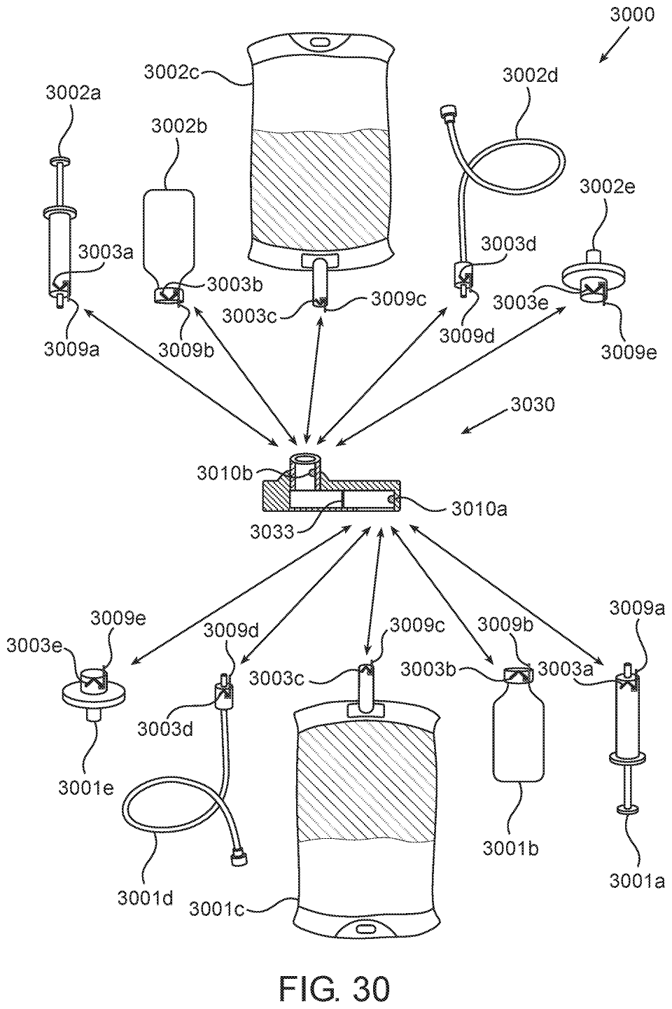

FIG. 30 illustrates system 3000 with a decontamination device, in the form of a connector 3030 that can be engaged with various types of first and second vessels. The device 3030 has a static wiping member 3033 that can decontaminate a surface of the first and/or second vessel upon movement thereof within the device 3030. As illustrated, first vessel may be, for example, a syringe 3001a, a bottle 3001b, a bag 3001c, an infusion line 3001d or a filter 3001e. As illustrated, second vessel may be, for example, a syringe 3002a, bottle 3002b, bag 3002c, infusion line 3002d or a filter 3002e. The first and/or second vessels, optionally at least a portion of first and/or second vessels, are moveable within the connector, thereby effecting decontamination of surfaces of the vessels. A valve 3003 is disposed within a port of first vessel and/or second vessel. A valve lock mechanism 3009 may be further disposed in first and/or second vessel(s) to lock the valve and maintain thereof locked prior to an engagement with device 3030. The device 3030 includes a valve unlock mechanism 3010 which can unlock valve lock mechanism when the device engages a vessel. The device 3030 is a decontamination device.

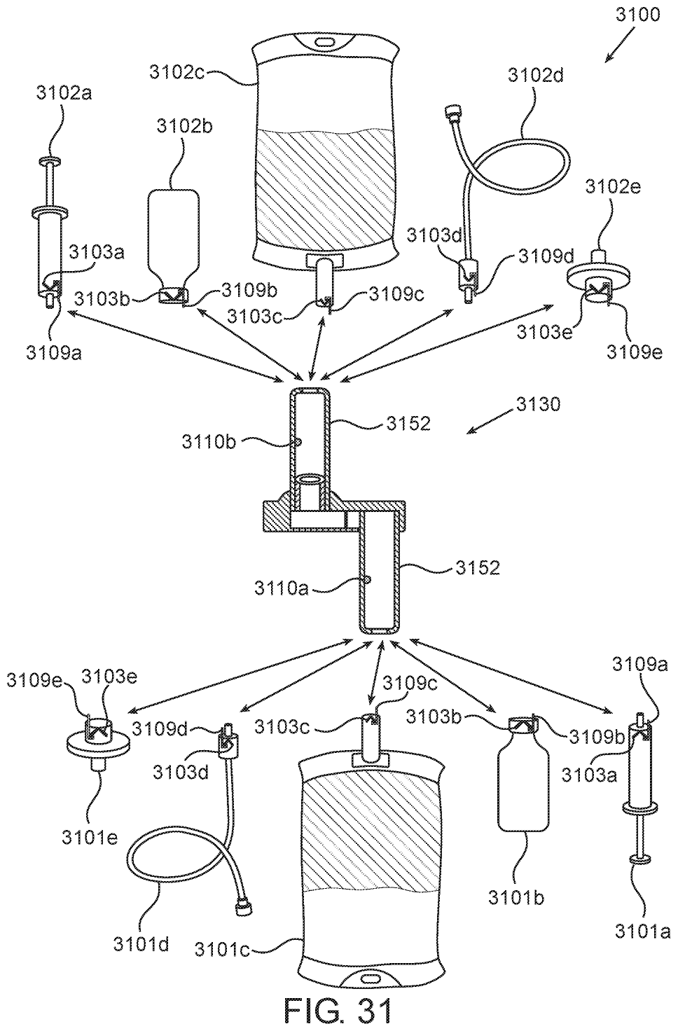

FIG. 31 illustrates yet a similar system, system 3100 wherein device 3130 includes downward extending walls 3152 at opposing sides thereof for enclosing a lumen for accommodating therein vessel 3101 and vessel 3102, or a portion thereof. A valve unlock mechanism 3110 is disposed in this case to extend internally from the downward/upward extending walls 3152.

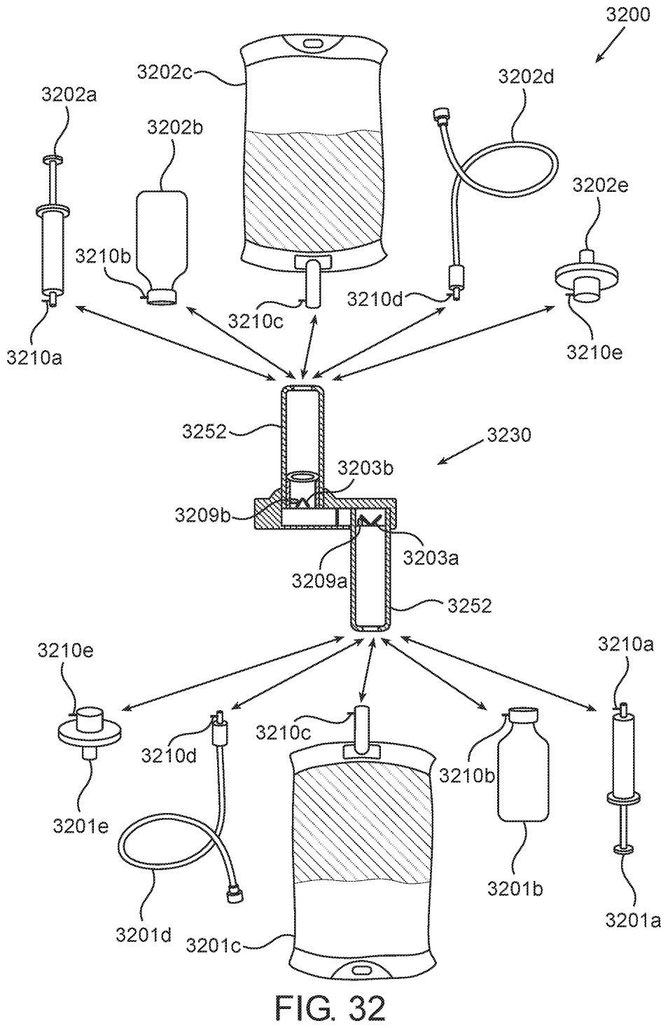

FIG. 32 In system 3200, the connector 3230 includes downward and upward extending walls 3252 and valves 3203a and 3203b closing apertures for receiving vessels of the device 3230. A valve lock mechanism 3209a and 3209b is further disposed within/on the device 3230 and provided to maintain the closed configuration of the valves 3203a and 3203b, respectively. A valve unlock mechanism 3210 is provided on each of a first and/ second vessels 3201 and/or 3202 to allow the unlocking of lock mechanism 3209a and/or 3209b, respectively.

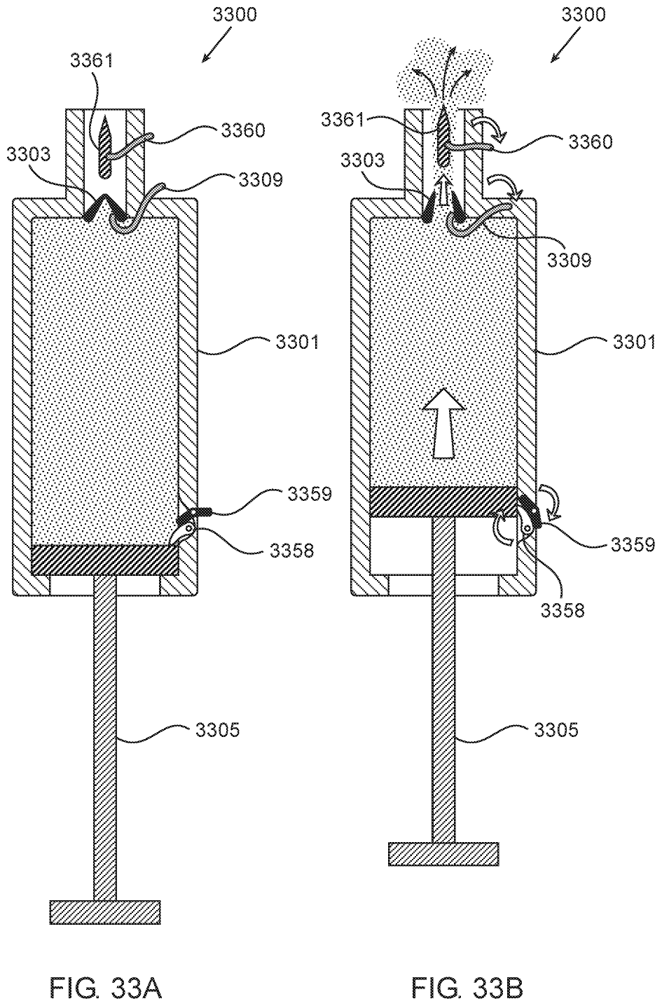

FIGS. 33A-33B illustrate yet another exemplary vessel, here syringe 3301 which as illustrated herein includes a valve 3303 and a valve lock mechanism 3309. Syringe 3301 further includes a piercing member 3361 to possibly pierce a membrane/covers/seal of vessel 3301 or of a second vessel (not shown). A piercing member lock mechanism 3360 is further incorporate to maintain the lock position of the piercing member 3361. Syringe 3301 further includes a plunger 3305, and a plunger lock mechanism 3358 in order to monitor the fluid transfer and/or prevent a premature fluid transfer. Plunger locking mechanism 3358 has a pin-like form, that prevents premature/accidental movement of the plunger 3305. The plunger lock mechanism 3358 is connected to an inner wall of the vessel 3301 and protrudes therefrom, nevertheless, alternative positions of the plunger lock mechanism may be applicable and are contemplated. The lock mechanism 3358 may extend from a socket within a wall of vessel 3301 and protrude inwardly into a lumen of the vessel. Optionally, the plunger lock mechanism 3358 is attached to a wall in syringe 3301 via a hinge which allows movement of the lock mechanism between a locked position and an unlocked retracted position. Syringe 3301 further includes a plunger unlocking mechanism 3359, having a pin-like form, for securing the lock position and mediating the locking-unlocking of the plunger 3305. FIG. 33A shows the syringe 3301 having a plunger lock mechanism, a valve lock mechanism, and a needle lock mechanism. However, syringe 3301 may have any combination of the lock mechanisms, for example a plunger lock mechanism and a valve lock mechanism, a plunger lock mechanism and a needle lock mechanism, and/or a valve lock mechanism and a needle lock mechanism. FIG. 33A shows the lock mechanisms in a locked state/position. FIG. 33B shows the plunger lock mechanism in an unlocked position which allows the plunger to move upwards towards the syringe tip, the valve lock mechanism in an unlocked position which opens the valve and allows a fluid to move into and/or out of the syringe, and a needle lock mechanism in an unlocked state which allows the needle to pierce through a surface of the syringe and/or another vessel (not shown). This unique combinations of a plurality of lock mechanisms provides safety for the user since if one of the lock mechanisms fails another lock mechanism will prevent premature spillage of a beneficial substance contained within the syringe into the environment.

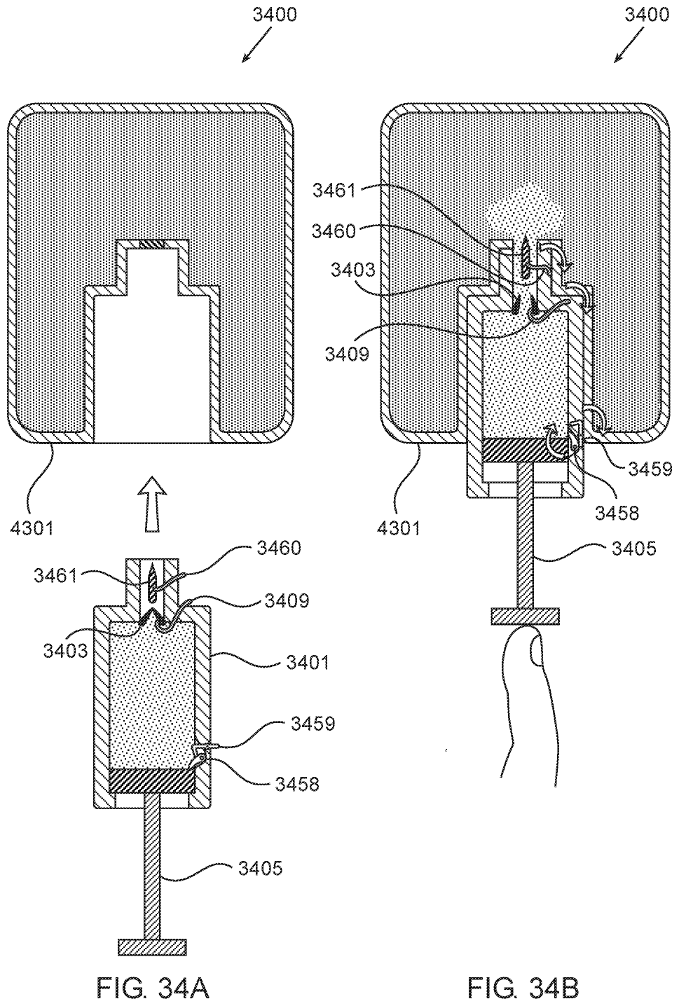

FIGS. 34A-34B illustrate a system 3400 which includes a first vessel a first medical vessel (syringe) 3401 and a second medical vessel (container) 4301, for transferring a beneficial/medical substance therebetween. The plunger lock mechanism 3458 and unlocking mechanism 3459 are hingedly connected to a side wall of a syringe 3401. The plunger unlocking mechanism 3459 blocks at least a portion of plunger lock mechanism 3458 and thereby locks locking mechanism 3458, maintaining same at the lock position (FIG. 34A). System 3400 further includes a valve 3403 and a valve lock mechanism 3409. The system 3400 further includes piercing member 3461 and a piercing member lock mechanism 3460. Upon entry of vessel 3401 into a lumen of vessel 4301, the valve lock mechanism 3409, piercing member lock mechanism 3460, and plunger lock mechanism 3459 are unlocked and retracted, allowing unlocking/releasing piercing member 3461 valve 3403 and plunger 3405. A user can then push plunger 3405 upwardly to thereby inject the beneficial substances/medicament into second vessel 4301 (FIG. 34B). The piercing member 3461 can pierce a membrane/seal or cover of first and/or second vessels 3401 and/or 4301. The valve 3403 allows the movement of a fluidic substance from first vessel 3401 into second vessel 4301.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for the engagement and establishment of fluid passageway between vessels, the system comprising:
a first vessel comprising a valve; and
a second vessel configured to be coupled to the first vessel;
wherein the second vessel comprises a valve unlock mechanism configured to unlock/open the valve, and wherein at about the time of or following the engagement between the first and second vessels, the valve unlock mechanism unlocks/opens the valve, allowing a fluid transfer between the vessels.

2. The system of claim 1, wherein the valve is a one-way valve or a two-way valve.

3. The system of claim 1, wherein the valve is selected from a ball valve, a butterfly valve, choke valve, diaphragm valve, a gate valve, a knife valve, a needle valve, a pinch valve, a piston valve, a plug valve, a solenoid valve, a spool valve, and a combination thereof.

4. The system of claim 1, further comprising a valve lock mechanism configured to lock the valve in a closed position and prevent the premature fluid transfer from the first vessel to the second vessel prior to an engagement with the second vessel.

5. The system of claim 4, wherein the valve lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

6. The system of claim 1, wherein the valve unlock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, a slot, and a combination thereof.

7. The system of claim 1, wherein the valve is pivotally connected to a side wall of the first and/or second vessel and moveable about said pivot.

8. The system of claim 1, wherein the valve lock and/or unlock mechanism is pivotally connected to a side wall of the first and/or second vessel and moveable about said pivot.

9. The system of claim 1, wherein the valve lock and/or unlock mechanism is spring loaded or attached to a spring, allowing planar movement thereof.

10. The system of claim 1, further comprising an engagement mechanism to connect the first vessel to the second vessel, wherein the engagement mechanism is selected from a thread, a luer, a ratchet teeth mechanism, a retention member, an adhesive mechanism, a clamping member and combinations thereof.

11. The system of claim 1, further comprising a piercing member to pierce a seal/cover of the first and/or second vessel(s).

12. The system of claim 11, further comprising a piercing member lock mechanism to prevent premature seal/cover piercing of the first vessel and/or second vessel, by the piercing member.

13. The system of claim 1, further comprising a plunger disposed on the first and/or second vessel(s), the plunger configured to allow for the fluid transfer from the first vessel into the second vessel.

14. The system of claim 13, further comprising a plunger lock mechanism configured to lock a plunger and prevent fluid transfer from the first vessel to the second vessel prior to an engagement with the second vessel.

15. The system of claim 1, wherein the second vessel includes a port having peripheral walls enclosing a cavity, the cavity configured to receive or engage with the first vessel, or a portion thereof.

16. The system of claim 1, further comprising a decontamination device configured to be coupled to the first vessel and second vessel and allow the fluid passageway therebetween, the decontamination device further configured to decontaminate a surface of at least one of the first and second vessels.

17. The system of claim 16, wherein the decontamination device includes a wiping member configured to wipe off a surface of a vessel, wherein the wiping member is configured to move within the device such to allow decontamination a surface of at least one of said first and second vessels at about the time of said wiping member's movement.

18. The system of claim 17, wherein the first and/or second vessel is configured to move within the device and wherein the wiping member configured to remove contaminants from a surface of said first and/or second vessels at about the time of said vessel's movement.

19. The system of claim 17, wherein the decontamination device includes a sliding mechanism located on and/or attached to said wiping member and/or said vessel and configured to allow for the movement of said wiping member and/or said vessel within said device.

20. The system of claim 19, further comprising a valve lock mechanism configured to lock the valve in a closed position and prevent the premature fluid transfer from the first vessel to the second vessel prior to an engagement with the second and wherein the valve unlock mechanism disposed on the decontamination device and/or on the first vessel.
